(19) United States
(12) Patent Application Publication
(10) Pub. No.: US 2026/0234557 A1
(43) Pub. Date: Aug. 13, 2026

(54) PLURIPOTENT STEM CELL-DERIVED T CELL POPULATIONS AND PROGENITORS THEREOF

(71) Applicant: Stratus Therapeutics, Inc., Cambridge, MA (US)

(72) Inventor: Dhvanit Shah, Chestnut Hill, MA (US)

(21) Appl. No.: 19/117,864

(22) PCT Filed: Oct. 5, 2023
(86) PCT No.: PCT/US23/76110

§ 371 Date: Apr. 2, 2025

Related U.S. Application Data
(60) Provisional application No. 63/413,338, filed on Oct. 5, 2022.

(51) Int. Cl. C12N 5/0783; A61K 35/17

(52) U.S. Cl. CPC C12N 5/0636; A61K 35/17; C12N 2506/45; C12N 2510/00

(57) ABSTRACT

In various aspects and embodiments, the present disclosure provides methods for generating T cell populations for cell therapy, including T lymphocytes (T cells) and progenitor T cells. In various embodiments, the invention provides for efficient ex vivo processes for developing progenitor T cells and T cell populations (including but not limited to CD4+CD8+ “double positive” T cells, as well as single positive CD4+ T helper cells and CD8+ cytotoxic T cells, and T regulatory cells) from human induced pluripotent stem cells (iPSCs). Cells generated according to the disclosure in various embodiments are functional and/or more closely resemble the corresponding lineage isolated from peripheral blood or lymphoid organs. The present invention in some aspects provides isolated cells and cell compositions produced by the methods disclosed herein, as well as methods for cell therapy.

### PRIORITY

This application claims priority to, and the benefit of. U.S. Provisional Application No. 63/413,338 filed Oct. 5, 2022, which is hereby incorporated by reference in its entirety.

### SEQUENCE LISTING

The instant application contains a Sequence Listing which has been submitted in XML format via EFS-Web and is hereby incorporated by reference in its entirety. Said XML copy, created on Sep. 25, 2023, is named GRU-016/121145-5016_Sequence_Listing.xml and is 30,062 bytes in size.

### BACKGROUND

The generation of hematopoietic cells from pluripotent cells ex vivo has attracted the interest of the scientific community for its prospects for allogeneic compatible cell-based therapies. Induced pluripotent stem cells (iPSCs) could potentially serve as a supply for generating “off-the-shelf” therapeutic lymphocytes. Nianias, A., & Themeli, M., Induced pluripotent stem cell (iPSC) derived lymphocytes for adoptive cell immunotherapy: recent advances and challenges. Current Hematologic Malignancy Reports, 14 (4), 261-268 (2019). However, significant hurdles remain in developing methods for making clinically relevant numbers of hematopoietic cell lineages, such as T cells, having clinically advantageous phenotypes. Accordingly, successful generation of T cell populations suitable for cell therapy ex vivo from iPSCs would fill a great need. In various aspects and embodiments, the invention meets these objectives.

### SUMMARY OF THE DISCLOSURE

The present disclosure, in various aspects and embodiments, provides methods for generating T cell populations for cell therapy, including T lymphocytes (T cells) and progenitor T cells. In various embodiments, the invention provides for efficient ex vivo processes for developing progenitor T cells and T cell populations (including but not limited to precursor T cells, CD4+CD8+ “double positive” T cells, CD4+ helper T cells, CD8+ cytotoxic T cells, and T regulatory cells) from human induced pluripotent stem cells (iPSCs). Cells generated according to the disclosure in various embodiments are functional and/or more closely resemble the corresponding lineage isolated from peripheral blood or lymphoid organs. The present invention in some aspects provides isolated cells and cell compositions, including cell compositions produced by the methods disclosed herein, as well as methods (and uses) for cell therapy.

In one aspect, the disclosure provides a method for preparing a T cell population or a population of T-progenitor cells. The method comprises enriching for CD34+ cells from a differentiated pluripotent stem cell population to prepare a CD34+-enriched population. Endothelial-to-hematopoietic transition (EHT) is induced in the CD34+-enriched population for at least two days, but no more than twelve days, to prepare a population comprising hematopoietic stem cells (HSCs) and/or hematopoietic stem progenitor cells (HSPCs). The resulting population comprising HSCs and/or HSPCs (or fraction thereof) is differentiated to a population comprising a T cell population or progenitor T cell population.

Conventionally, hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with aspects and embodiments of this disclosure, it is discovered that inducing endothelial-to-hematopoietic transition (EHT) of a CD34+ cell population, and which can be derived from iPSCs-embryoid bodies, can be used for the ex vivo generation of superior T progenitor cell and T cell populations.

In a non-limiting example, to generate mature T cells, embryoid body formation is used to generate CD34+ cells, from which HSCs and/or HSPCs are derived by inducing EHT. The HSC and/or HSPC population are then cultured in a T cell media supplemented with, for example, retroNectin and DLL-4 for the generation of Tpro cells or pre-T cells. Further culturing will generate mature T cells. Foxp3 expression in α/β T cells results in Treg generation ex vivo. Cells can optionally be harvested or recovered at certain steps, or in some embodiments, differentiation to the desired T cell population does not comprise a harvesting/recovery step. That is, the differentiation can take place continuously in culture.

In some aspects and embodiments, this disclosure provides a method for generating a CD7+ progenitor T cell population, or a derivative of this population. For example, the method comprises generating an HSC and/or HSPC population, which can comprise human long-term hematopoietic stem cells (LT-HSCs) from iPSCs (e.g., hiPSCs). The HSC and/or HSPC population is derived by induction of endothelial-to-hematopoietic transition of CD34+ cells (e.g., CD34+ cells derived from embryoid bodies). The HSC and/or HSPC population (or cells isolated therefrom) is cultured with a partial or full Notch ligand, sonic hedgehog (SHH), RetroNectin (or other extracellular matrix component(s)), and/or combinations thereof, to produce a population comprising CD7+ progenitor T cells or a derivative cell population (e.g., a T cell population).

In various embodiments, the iPSCs are prepared by reprogramming somatic cells, such as but not limited to CD34+ cells isolated from peripheral blood. In various embodiments, the iPSCs are autologous or allogenic (e.g., HLA-matched at one or more loci) with respect to a recipient. In various embodiments, the iPSCs can be gene edited to assist in HLA matching. For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I and at least one HLA Class II complex. In some embodiments, T cell populations are derived from iPSCs that are gene edited to be HLA-A^{neg}, homozygous for both HLA-B and HLA-C, and HLA-DPB1^{neg} and HLA-DQB1^{neg}. In some embodiments, the iPSCs are further homozygous for HLA-DRB1.

In some embodiments, the process according to aspects can comprise generating CD34+-enriched cells from the differentiated pluripotent stem cells (e.g., from EBs) and inducing endothelial-to-hematopoietic differentiation. HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means. In some embodiments, CD34+ enrichment and EHT are induced once the cells are at least 20% CD34+, for example, may be induced at Day 6 to Day 14 of iPSC differentiation, such as for example, Day 8, Day 9, Day 10, Day 11, Day 12, Day 13, or Day 14. Differentiation of iPSCs can be according to known techniques.

Induction of EHT can be with any known process. In various embodiments, EHT can be induced in the culture for from 2 days to 12 days. In some embodiments, EHT is induced in the culture from about 5 days to about 7 days. In some embodiments, the method comprises increasing activity or expression of DNA (cytosine-5-)-methyltransferase 3 beta (Dnmt3b) in a cell population, such as a CD34+ cell population comprising hemogenic endothelial cells. In some embodiments, cells are contacted with an effective amount of an agonist of a mechanosensitive receptor or a mechanosensitive channel that increases the activity or expression of Dnmt3b. In some embodiments, the mechanosensitive receptor is Piezol. An exemplary, non-limiting Piezol agonist is Yoda1.

In various embodiments, CD34+ cells are harvested from the culture undergoing endothelial-to-hematopoietic transition between Day 10 to Day 20 of iPSC differentiation, such as from Day 10 to Day 20, or from Day 12 to Day 15 of iPSC differentiation.

In various embodiments, the population comprising HSCs and/or HSPCs or fraction thereof is differentiated to a population comprising T progenitor cells or T lymphocytes. In some embodiments, the cell population is cultured with a Notch ligand, partial or full, SHH, extracellular matrix component(s), and/or combinations thereof, ex vivo, to differentiate HSCs to T cell population (or precursor thereof). Differentiation to progenitor T cells can further include in some embodiments the presence of stem cell factor (SCF), Flt3L and interleukin (IL)-7. For example, HSCs and/or HSPCs can be cultured in a medium comprising TNF-α, IL-7, thrombopoietin (TPO), Flt3L, and stem cell factor (SCF), and optionally SR1, in the presence of an immobilized Delta-Like-4 ligand and a fibronectin fragment. In some embodiments, cells are cultured for 7 to 14 days to prepare progenitor T cells or pre-T cells (e.g., CD34−, CD7+, CD5+/−). In some embodiments, cells are cultured for 15 to 28 days for production of mature T cells (e.g., CD3+), including optionally production of Tregs. In some embodiments, T lymphocytes and progenitor T cells can be differentiated to Tregs by expression of FOXP3, and which are optionally expanded in culture.

In still other embodiments, the present invention generates T cells (e.g., CTLs, helper T cells, or Tregs) expressing a chimeric antigen receptor. Cells can be efficiently transduced by a vector, such as but not limited to retroviral or nonintegrating viral vectors, nonviral vectors, and episomal or episomal hybrid vectors carrying a CAR targeting tumor antigen, including but not limited to CD19, CD38, CD33, CD47, and CD20 etc. CARs can be designed to enhance a cells ability to recognize, bind to, and/or kill target cells. In some embodiments, the CAR enhances the cell's ability to recognize tumor cells. In some embodiments, the CAR enhances the cells anti-tumor activity.

In some embodiments, the present invention generates T cells that exhibit T cell activation and subsequent T-cell mediated cytotoxicity. The T cells generated herein can exhibit a significant outperformance in T cell-mediated cytotoxicity in comparison to CD34+ derived T cells.

In other aspects, the invention provides a cell population, or pharmaceutically acceptable composition thereof, produced by the method described herein. In some embodiments, the cell population is a progenitor T cell population capable of engraftment in a thymus, spleen, or secondary lymphoid organ upon administration to a subject in need. In other embodiments, the cell population is an α/β T cell population, a CAR-T cell population, a CTL population (which can express a CAR), a T helper population, or a Treg population (each as described herein).

In some embodiments, the cell population is a Treg population useful for adoptive cell therapy, for example, for human subjects having a condition selected from an autoimmune or inflammatory condition or disease or graft versus host disease (GVHD). Further, various genetic disorders can impact the immune system, manifesting as autoimmune or pro-inflammatory state. In some embodiments, the Treg population is a CAR-T cell, expressing a tissue or cell-specific CAR.

In another aspect, the disclosure provides a cell composition, which comprises a T cell population (or progenitor thereof, such as a progenitor T cell population) that is HLA-A^{neg}, HLA-DPB1^{neg}, and HLA-DQB1^{neg}. Despite such gene deletions and/or gene edits, the T cell composition retains antigen presenting functionality comparable to non-edited cells, and the ability to differentiate from precursors (as described herein) as well as to more mature T cell phenotypes. Cell compositions of this aspect provide advantages in HLA matching for a recipient, to avoid, for example, GVHD. In various embodiments, the T cell population is homozygous for both HLA-B and HLA-C. In some embodiments, the T cell population is homozygous for HLA-DRB1.

In some embodiments of this aspect, the T cell population is a T-progenitor cell population. In various embodiments, the T-progenitor cell population engrafts in the thymus or spleen. In other embodiments, the T cell population is a cytotoxic T cell (CTL) population, a helper T cell population, or a Treg population. In various embodiments, the T cell population may express a chimeric antigen receptor (CAR). The cell populations according to this aspect may be prepared according to other aspects of this disclosure.

In other aspects, the invention provides a method for cell therapy (or uses of the cell compositions for cell therapy), comprising administering a cell population described herein, or pharmaceutically acceptable composition thereof, to a human subject in need thereof. In various embodiments, the methods described herein are used to treat blood (malignant and non-malignant), bone marrow, and immune diseases. In various embodiments, the human subject has a condition comprising one or more of lymphopenia, a cancer, infectious disease (e.g., viral disease such as HPV or HIV) an immune deficiency, an autoimmune disease, a skeletal dysplasia, hemoglobinopathies, an anemia, a bone marrow failure syndrome, and a genetic disorder (e.g., a genetic disorder impacting the immune system).

In some embodiments, the subject has cancer, such as a hematological malignancy or a solid tumor. In such embodiments, the subject is administered T-progenitor cells, or T cells having an anti-tumor specificity (such as CTLs recognizing tumor antigens).

In other embodiments, the invention provides a method for cell therapy, comprising administering a Treg cell population described herein, or pharmaceutically acceptable composition thereof, to a human subject in need thereof. In various embodiments, the subject has an autoimmune, alloimmune, or inflammatory disease. In some embodiments, the subject is a tissue or organ transplant recipient, and in some embodiments the subject is a recipient of an allogeneic organ or tissue transplant. In some embodiments, the subject is experiencing or is at risk for GVHD. Organs that can be transplanted, for example, include the heart, kidneys, liver, lungs, pancreas, intestine, and thymus, among others. Tissues for transplant can include, for example, bones, tendons (both referred to as musculoskeletal grafts), bone marrow or HSCs, cornea, skin, heart valves, nerves and/or veins.

In some embodiments, the subject has an autoimmune condition, which in some embodiments is selected from Type 1 diabetes, Rheumatoid arthritis (RA), Psoriasis or psoriatic arthritis, Multiple sclerosis, Systemic lupus erythematosus (SLE), Inflammatory bowel disease, Addison's disease, Graves' disease, Sjogren's Syndrome, Hashimoto's thyroiditis, Myasthenia gravis, autoimmune vasculitis, scleroderma, Hemolytic anemia, Pernicious anemia, and Goodpasture's syndrome.

Other aspects and embodiments of this disclosure will be apparent from the following detailed disclosure and working examples.

### BRIEF DESCRIPTION OF DRAWINGS

FIG. 1 shows that ETV2 over-expression (OE) does not affect pluripotency. FIG. 1 shows FACS plots representative of transduction efficiency of iPSC with an adenoviral vector to overexpress ETV2 and GFP sequences. ETV2 overexpression does not affect the iPSC stemness as shown by the expression of the TRA-1-60 stemness marker.

FIG. 2 shows that ETV2 over-expression (OE) increases the yield of hemogenic endothelial cells. Representative flow cytometric analysis of hemogenic endothelial cells (described as CD235a−CD34+CD31+) and relative quantification demonstrates that ETV2-OE enhances the formation of hemogenic endothelial cells.

FIG. 3 shows that ETV2 over-expression (OE) enhances CD34+ cell formation during iPSC differentiation. Representative flow cytometric analysis of CD34+ cells and relative quantification demonstrates that ETV2-OE enhances the CD34+ cell formation.

FIG. 4A and FIG. 4B show that iPSC-derived HSCs that are derived with Piezo1 activation undergo pro-T cell differentiation similar to bone marrow (BM)-HSCs. FIG. 4A is a FACS plot of differentiation efficiency to CD34+CD7+ pro T cells of Bone Marrow (BM) HSCs and iPSC-HSCs derived with Piezol activation. FIG. 4B is a quantification of CD34+CD7+ cells (%) derived with (1) BM-HSCs and (2) iPSC-HSCs (Piezo1 Activation). FIG. 4B shows the average of three experiments.

FIG. 5A and FIG. 5B show that iPSC-derived HSCs generated with Piezo1 activation undergo T cell differentiation and such T cells can be activated with CD3/CD28 beads similar to T cells derived from BM-HSCs. FIG. 5A is a FACS plot of activation efficiency (CD3+CD69+ expression) of T cells differentiated from BM-HSCs and iPSC-derived HSCs generated with Piezo1 activation. FIG. 5B is a quantification of CD3+CD69+ cells (%) derived with (1) BM-HSCs and (2) iPSC-HSCs (Piezo1 Activation). FIG. 5B shows the average of three experiments.

FIG. 6 shows that iPSC-derived HSCs (in this example with Piezo1 activation) can differentiate to functional T cells. IFNγ expression is a consequence of T cell activation after T cell receptor (TCR) stimulation via CD3/CD28 beads. IFNγ expression in T cells differentiated from iPSC-derived HSCs, generated upon Piezo1 activation, enhances HSC ability to further differentiate to functional T cells. FIG. 6 shows the average of three experiments.

FIG. 7 shows that HSCs generated according to this disclosure (D8+7 iPSC-CD34+ cells, with and without Yoda 1, “Y”) successfully differentiate into CD4+CD8+ (“double positive”) T cells as well as TCR α/β T cells. The methods of the present disclosure substantially outperform T cell maturation from bone marrow CD34+ cells.

FIG. 8 shows that HSCs generated according to this disclosure (D8+7 iPSC-CD34+ cells, with or without Y) successfully rearrange TCR, and outperform bone marrow CD34+ cells.

FIG. 9A and FIG. 9B show the phenotype analysis of HLA edited (e.g., triple knockout) cells performed by FACS and immunofluorescence. FIG. 9A shows the overall expression of HLA class-I molecules (HLA-A, HLA-B, and HLA-C) on the cell surface, where the HLA edited cells are positive for overall HLA class-I expression to a similar degree as wild-type cells (gHSCs). FIG. 9B shows cell expression of HLA-A via immunofluorescence, where HLA-A is not expressed in the HLA edited clone.

FIG. 10 shows that the HLA edited clones preserve their pluripotency (maintain trilineage differentiation), as illustrated by immunofluorescence, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

FIG. 11 shows the immune compatibility of the HLA edited HSCs. HLA edited HSCs and control HSCs (WT, B2M KO, and HLA Class II null) were co-cultured with peripheral blood mononuclear cells (PBMCs) matching HLA-B and HLA-C, but with mismatched HLA-A. The PBMC-mediated cytotoxicity was measured by an annexin V staining assay.

FIG. 12 shows in vivo engrafting potential of HLA edited HSCs. Equal proportions of mCherry HLA edited HSCs and wild-type HSCs (gHSCs) were mixed for a competitive transplant into mice, where bone marrow (BM) and peripheral blood samples were evaluated by FACS to compare the relative amounts of each cell type present in the samples.

FIGS. 13A and 13B show that deletion of HLA-A does not impact Class I peptide presentation. FIG. 13A shows a schematic representation of immunopeptidome analysis.

FIG. 13B shows results of the immunopeptidome analysis, which reveals that little difference exists in the numbers of peptides and representative proteins presented by class I molecules of WT and HLA-edited cells.

FIGS. 14A and 14B show that deletion of HLA-DP and DQ does not impact Class II peptide presentation. FIG. 14A shows immunopeptidome analysis scheme. FIG. 14B shows that despite the deletion of HLA-DP and DQ, the cells preserve their ability to present a broad spectrum of peptide through HLA Class II.

FIG. 15 is a schematic representation of in vivo testing of antigen-mediated immune response: Delayed Type Hypersensitivity Assay (DTH), sensitizing stage and elimination stage respectively.

FIGS. 16A and 16B show that HLA-edited HSCs reconstitute a functional immune system as demonstrated by DTH reaction in immune deficient mice. FIG. 16A shows a delayed-type hypersensitivity assay on transplanted mice were performed, which is an assay that involves the cross-talk of different types of immune cells. Mice were sensitized by subcutaneous injection of sheep Red blood cells (antigen). A functional immune system results in the swelling of the left paw that was measured with a micro caliper. As can be seen in FIG. 16A, the non-transplant mice did not show any left paw swelling as they are immunodeficient. Conversely, the mice transplanted with Cord Blood CD34+ cells show tissue swelling and doubled the diameter of their left paw. FIG. 16B is a graphical evaluation of the results shown in FIG. 16A.

FIG. 17 shows the HSC differentiation potential into T cell subtypes. After a 35-day differentiation period pro-T cells were evaluated by cell sorting for the presence of CD4+, CD8+, and AB+ T cell populations. FIG. 17 compares the differentiation potential of bone marrow-derived CD34+ cells, embryoid body CD34+ cells, and HSCs prepared according to the present disclosure (e.g., using Piezo1 activation) (“gHSCs”).

FIG. 18 shows the degree of T-cell mediated cytotoxicity measured from a co-culture of HSC-derived T cells with CD19+ lymphoma cells in the presence of an anti-CD3/CD-19 bispecific antibody. T cells prepared from HSCs according to the present disclosure (“gHSC”) demonstrate a high level of cytotoxicity against the target cells.

FIG. 19 shows that HSC-derived T cells (pro-T cells) can be transduced with high efficiency. Pro-T cells underwent lentiviral (LV) transduction with an anti-CD-19 chimeric antigen receptor (CAR) transgene (left), where the efficiency of LV transduction was measured by cell sorting based on anti-CD19 scFv staining (right). Results indicate that HSC-derived T cells achieved approx. 85% transduction efficiency.

FIG. 20 shows that LV-transduced HSC-derived T cells (pro-T cells) can effectively mature into CD4+/CD8+ T cells via CAR transduction.

FIG. 21 shows the ability of anti-CD19 CAR-transduced HSC-derived T cells (CAR pro-T cells) to function via receptor-mediated cytotoxicity. Luc+ NALM6 leukemia cells were co-cultured with CAR pro-T cells and cell-mediated cytotoxicity was measured by luciferase assay.

FIG. 22 shows the ability of the HSCs to develop into pro-T cells as measured by their CD34-CD7+markers.

FIGS. 23A and 23B demonstrates increased expression of T cell-specific transcription factors and Thymus engrafting molecules with the pro-T cells derived from HSCs according to the instant disclosure. FIG. 23A shows TCF7 mRNA expression and FIG. 23B shows CCR7 mRNA expression.

FIGS. 24A and 24B shows that HSC-derived Pro-T Cells engraft and differentiate in thymus. FIG. 24A illustrates the engraftment and analysis procedure. FIG. 24B shows FACS analysis of CD3 cell population of cells gated on CD45+ cell population, which shows the superior engraftment and differentiation potential of the HSC-derived Pro-T Cells in the thymus.

FIG. 25 shows HSC-derived T cells can be activated in vitro. Top panel shows FACS analysis of activated T cells from different sources, including from HSCs prepared according to the present disclosure. T cells of the present disclosure demonstrate comparable or superior activation as measured by increased CD107 expression. The lower panel shows Dynabeads activation, where activated T cells express inflammatory cytokines. HSC-derived T cells express higher levels of inflammatory cytokines as exemplified by TNF-alpha and interferon gamma expression levels.

FIG. 26 shows that CCR5-knocked out HSCs can comparably differentiate into pro-T cells, compared to their wild type (gHSC) counterpart HSC (CCR5 retained).

FIG. 27 shows CCR5-knocked out HSCs can comparably differentiate into double positive (CD4+CD8+) T cells when compared to their wild type (gHSC) counterpart HSCs (CCR5 retained).

The term “gHSC” is used herein to refer to the iPSC-derived hematopoietic stem cells of the present disclosure.

The terms “wild type” (WT), “unedited”, “non-HLA-edited” are used interchangeability herein to refer to the non-gene edited cells of the present disclosure.

EB34+ cells refer to Embryonic body derived CD34+ cells. These comprise hemogenic endothelial cells.

### DESCRIPTION OF THE INVENTION

The present disclosure, in various aspects and embodiments, provides methods for generating T cell populations for cell therapy, including T lymphocytes (T cells) and progenitor T cells. In various embodiments, the invention provides for efficient ex vivo processes for developing progenitor T cells and T cell populations (including but not limited to precursor T cells, CD4+CD8+ “double positive” T cells, CD4+ helper T cells, CD8+ cytotoxic T cells, and T regulatory cells) from human induced pluripotent stem cells (iPSCs). Cells generated according to the disclosure in various embodiments are functional and/or more closely resemble the corresponding lineage isolated from peripheral blood or lymphoid organs. The present invention in some aspects provides isolated cells and cell compositions, including those produced by the methods disclosed herein, as well as methods for cell therapy.

In accordance with aspects and embodiments of this disclosure, the ability of human induced pluripotent stem cells (hiPSCs) to produce essentially limitless pluripotent stem cells (PSCs) is leveraged to generate a boundless supply of T cell populations or precursors thereof. Use of primary T cells as therapeutic lymphocytes has been limited by their restricted availability, cell numbers, limited expansion potential, and histocompatibility issues. For example, Tregs are present at low numbers in circulation and are challenging to isolate and expand ex vivo. Moreover, compared to primary cells, hiPSCs can more readily undergo genetic modifications in vitro, thereby offering opportunities to improve cell-target specificity, cell numbers, as well as bypassing HLA-matching issues for example. Additionally, fully engineered hiPSC clones, as compared to primary cells, can serve as a stable and safe source (Nianias and Themeli, 2019). Further, because hiPSCs, unlike human Embryonic Stem Cells (hESCs), are of non-embryonic origin, they are also free of ethical concerns. Accordingly, use of hiPSCs according to this disclosure confers several advantages over primary cells to generate therapeutic numbers of T cells or progenitors, including antigen-specific or tissue-specific T cells (including Tregs).

In one aspect, the disclosure provides a method for preparing a T cell population or a population of T-progenitor cells. The method comprises enriching for CD34+ cells from a differentiated pluripotent stem cell population to prepare a CD34+-enriched population. Endothelial-to-hematopoietic transition (EHT) is induced in the CD34+-enriched population for at least two days, but no more than twelve days, to prepare a population comprising hematopoietic stem cells (HSCs) and/or hematopoietic stem progenitor cells (HSPCs). In various embodiments, the HSC and/or HSPC population is a non-adherent cell population. In some embodiments, these cells are further enriched for CD34+ cells. The resulting population comprising HSCs and/or HSPCs (or fraction thereof) is differentiated to a population comprising a T cell population or progenitor T cell population.

Conventionally, hematopoietic lineages are prepared by differentiation of iPSCs to embryoid bodies up to day 8 to harvest CD34+ cells. CD34 is commonly used as a marker of hemogenic endothelial cells, hematopoietic stem cells, and hematopoietic progenitor cells. In accordance with aspects and embodiments of this disclosure, it is discovered that inducing endothelial-to-hematopoietic transition (EHT) of a CD34+ cell population, and which can be derived from iPSCs-embryoid bodies, can be used for the ex vivo generation of superior T progenitor cell and T cell populations, including but not limited to T regulatory cell (Tregs).

In a non-limiting example, to generate mature T cells, embryoid body formation, which takes about 8 to about 14 days (without limitation), is used to generate CD34+ cells, from which HSCs and/or HSPCs are derived by inducing EHT. The HSC and/or HSPC population (e.g., CD34+ cells undergoing EHT) are then cultured in a T cell media supplemented with, for example, retroNectin and DLL-4 for the generation of Tpro cells, identified as CD34+CD7+CD5+/− or pre-T cells, which can be identified as CD34−CD7+CD5+. Further culturing will generate mature T cells (CD3+, α/β T cells). Foxp3 expression in α/β T cells results in Treg generation ex vivo.

For example, the earliest intrathymic progenitors express high levels of CD34 and CD7, do not express CD1a, and are triple-negative (TN) for mature T cell markers: CD4, CD8, and CD3. Commitment to the T cell lineage is associated with the expression of CD1a by CD7-expressing pro-thymocytes. Thus, immature stages of T-cell development are typically delineated as CD34⁺CD1a⁻ (most immature) and CD34⁺CD1a⁺ cells. The transition from CD34⁺CD7⁺CD1a⁻ to CD34⁺CD7⁺CD1a⁺ by early thymocytes is associated with T-cell commitment. CD34⁺CD7⁺CD1a⁺ cells are likely T-lineage restricted. Following this stage, thymocytes progress to a CD4 immature single positive stage, at which point CD4 is expressed in the absence of CD8. Thereafter, a subset of the cells differentiates to the CD4⁺CD8⁺ double positive (DP) stage. Finally, following TCRα rearrangement, TCRαβ-expressing DP thymocytes undergo positive and negative selection, and yield CD4⁺CD8⁻ and CD4⁻CD8⁺ single positive (SP) T-cells.

In various embodiments, as described in detail herein, the population comprising HSCs and/or HSPCs is differentiated to a population comprising one or more of T-progenitor cells, precursor T cells, double positive T cells, single positive T cells (such as CD8+ or CD4+), and regulatory T cells (Tregs). Cells can optionally be harvested or recovered at certain steps, or in some embodiments, differentiation to the desired T cell population does not comprise a harvesting/recovery step. That is, the differentiation can take place continuously in culture.

In some embodiments, the population comprising HSCs and/or HSPCs is differentiated to a population comprising T-progenitor cells. T-progenitor cells can be recovered from the culture for cell therapy or alternatively further differentiated in culture. In some embodiments, the T-progenitor cells are further differentiated without first recovering them from the culture. For example, the T-progenitor cells may be further differentiated to a population comprising one or more of precursor T cells, double positive T cells (CD4+CD8+), single positive T cells (CD4+CD8− or CD8+CD4−), or regulatory T cells. In some embodiments, the T-progenitor cells are further differentiated to a population comprising regulatory T cells. The progenitor T cells are optionally recovered from the culture prior to such differentiation.

In some embodiments, the population comprising HSCs and/or HSPCs is differentiated to a population comprising double positive T cells (CD4+CD8+) and/or single positive T cells (CD4+CD8−; CD8+CD4−), which can optionally take place in continuous culture (i.e., without harvesting or recovering an intermediate cell population). In various embodiments, the double positive cells and/or single positive T cells are differentiated to regulatory T cells, optionally comprising a step of recovering double positive cells and/or single positive cells from the culture prior to differentiation to regulatory T cells.

In some embodiments, the population comprising HSCs and/or HSPCs is differentiated to a population comprising T regulatory cells, which can optionally take place in continuous culture (i.e., without harvesting or recovering an intermediate cell population).

In some aspects and embodiments, this disclosure provides a method for generating a CD7+ progenitor T cell population, or a derivative of this population. For example, the method comprises generating an HSC and/or HSPC population, which can comprise human long-term hematopoietic stem cells (LT-HSCs) from iPSCs (e.g., hiPSCs). The HSC and/or HSPC population is derived by induction of endothelial-to-hematopoietic transition of CD34+ cells (e.g., CD34+ cells derived from embryoid bodies). The HSC and/or HSPC population (or cells isolated therefrom) is cultured with a partial or full Notch ligand, sonic hedgehog (SHH), RetroNectin (or other extracellular matrix component(s)), and/or combinations thereof, to produce a population comprising CD7+ progenitor T cells or a derivative cell population (e.g., a T cell population).

The Notch signaling pathway regulates the formation, differentiation, and function of progenitor T-cells, pre-T cells, and/or mature T lymphocytes. In vivo, T cell development proceeds after lymphocyte progenitors differentiate from bone marrow hematopoietic stem cells and migrate to the thymus. Specialized thymic epithelial cells induce T cells to develop along a controlled pathway. Notch signaling plays a critical role during T lineage commitment in the thymus. As lymphoid progenitors enter the thymus, they encounter dense expression of Notch ligands on thymic epithelium that drives thymopoiesis. The present disclosure provides HSC and/or HSPC populations generated ex vivo from iPSCs and which respond to Notch ligand, SHH, and/or component(s) of extracellular matrix, by robust production of T progenitor cells and T cell lineages ex vivo.

In various embodiments, the iPSCs are prepared by reprogramming somatic cells. The term “induced pluripotent stem cell” or “iPSC” refers to cells derived from somatic cells, such as skin or blood cells that have been reprogrammed back into an embryonic-like pluripotent state. In some embodiments, iPSCs are generated from somatic cells such as (but not limited to) fibroblasts or PBMCs (or cells isolated therefrom). In some embodiments, the iPSCs are derived from lymphocytes (e.g., T-cells, B-cells, NK-cells, etc.), cord blood cells (including from CD3+ and/or CD4+ cells from cord blood), PBMCs, CD34+ cells, or other human primary tissues. In some embodiments, iPSCs are derived from CD34+ cells isolated from peripheral blood. In various embodiments, the iPSCs are autologous or allogenic (e.g., HLA-matched at one or more loci) with respect to a recipient (a subject in need of treatment as described herein). In various embodiments, the iPSCs can be gene edited to assist in HLA matching (such as deletion of one or more HLA Class I and/or Class II alleles or their master regulators, including but not limited beta-2-microglobulin (B2M), CIITA, etc.), or gene edited to delete or express other functionalities. For example, iPSCs can be gene edited to delete one or more of HLA-A, HLA-B, and HLA-C, and to delete one or more of HLA-DP, HLA-DQ, and HLA-DR. In certain embodiments, the iPSCs retain expression of at least one HLA Class I and at least one HLA Class II complex.

In various embodiments, T cell populations are derived from iPSCs which are gene edited to be one of: (i) HLA-A⁺B⁺C⁺DP⁻DR⁺DQ⁺, (ii) HLA-A⁻B⁺C⁺DP⁺DR⁺DQ⁻, (iii) HLA-A⁻B⁺C⁺DP⁻DR⁺DQ⁻; (iv) HLA-A⁻B⁻C⁺DP⁻DR⁺DQ⁺; (v) HLA-A⁻B⁻C⁺DP⁺DR⁺DQ⁻, (vi) HLA-A⁻B⁻C⁺DP⁻DR⁺DQ⁺. For retained HLA (for example HLA-B, HLA-C, and HLA-DR), cells can be homozygous or retain only a single copy of the gene. For example, the modified cells are identified at least as (a) HLA-C+ and HLA-DR+, and optionally identified as one or more of (b) HLA-B−, (c) HLA-DP−, and (d) HLA-DQ−. In exemplary embodiments, the modified cells are HLA-B+, HLA-DP−, and HLA-DQ−.

In some embodiments, T cell populations are derived from iPSCs that are gene edited to be HLA-A^{neg}, homozygous for both HLA-B and HLA-C, and HLA-DPB1^{neg} and HLA-DQB1^{neg}. In some embodiments, the iPSCs are further homozygous for HLA-DRB1.

As used herein, the term “neg,” (−), or “negative,” with respect to a particular HLA Class I or Class II molecule indicates that both copies of the gene have been disrupted in the cell line or population, and thus the cell line or population does not display significant functional expression of the gene. Such cells can be generated by full or partial gene deletions, or alternatively with other technologies such as siRNA. As used herein, the term “delete” in the context of a genetic modification of a target gene (i.e., gene edit) refers to abrogation of functional expression of the corresponding gene product (i.e., the corresponding polypeptide). Such gene edits include full or partial gene deletions, or deletions of critical cis-acting expression control sequences.

In some embodiments, the iPSCs are gene edited using gRNAs that are 16, 17, 18, 19, 20, 21, 22, 23, 24, 25, 26 or more nucleotides in length. In some embodiments, the gRNAs comprise a modification at or near the 5′ end (e.g., within 1-10, 1-5, or 1-2 nucleotides of the 5′ end) and/or a modification at or near the 3′ end (e.g., within 1-10, 1-5, or 1-2 nucleotides of the 3′ end). In some embodiments, the modified gRNAs exhibit increased resistance to nucleases. In some embodiments, a gRNA comprises two separate RNA molecules (i.e., a “dual gRNA”). A dual gRNA comprises two separate RNA molecules: a “crispr RNA” (or “crRNA”) and a “tracr RNA” and is well known to one of skill in the art.

Generally, various gene editing technologies are known, which can be applied according to various embodiments of this disclosure. Gene editing technologies include but are not limited to zinc fingers (ZFs), transcription activator-like effectors (TALEs), etc. Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducted using CRISPR associated Cas system (e.g., CRISPR-Cas9), as known in the art. See, for example, U.S. Pat. Nos. 8,697,359, 8,906,616, and 8,999,641, each of which is hereby incorporated by reference in its entirety. In various embodiments, the gene editing employs a Type II Cas endonuclease (such as Cas9) or employs a Type V Cas endonuclease (such as Cas12a). Type II and Type V Cas endonucleases are guide RNA directed. Design of gRNAs to guide the desired gene edit (while limiting or avoiding off target edits) is known in the art. See, for example, Mohr S E, et al., CRISPR guide RNA design for research applications, FEBS J. 2016 September; 283 (17): 3232-3238. In still other embodiments, non-canonical Type II or Type V Cas endonucleases having homology (albeit low primary sequence homology) to S. pyogenes Cas9 or Prevotella and Francisella1 (Cpf1 or Cas12a) can be employed. Numerous such non-canonical Cas endonucleases are known in the art. Nidhi S, et al. Novel CRISPR-Cas Systems: An Updated Review of the Current Achievements, Applications, and Future Research Perspectives, Int J Mol Sci. 2021 April; 22 (7): 3327. In still other embodiments, the gene editing employs base editing or prime editing to incorporate mutations without instituting double strand breaks. See, for example, Antoniou P, et al., Base and Prime Editing Technologies for Blood Disorders, Front. Genome Ed., 28 Jan. 2021; Matsuokas I G, Prime Editing: Genome Editing for Rare Genetic Diseases Without Double-Strand Breaks or Donor DNA, Front. Genet., 9 Jun. 2020. Various other gene editing processes are known, including use of dead Cas (dCas) systems (e.g., Cas fusion proteins) to target DNA modifying enzymes to desired targets using the dCas as a guide RNA-directed system. Brezgin S, Dead Cas Systems: Types, Principles, and Applications, Int J Mol Sci. 2019 December; 20 (23): 6041.

Base editors that can install precise genomic alterations without creating double-strand DNA breaks can also be used in gene editing (e.g., designing gene therapy vectors) in the cells (e.g., iPSCs). Base editors essentially comprise a catalytically disabled nuclease, such as Cas9 nickase (nCas9), which is incapable of making DSBs and is fused to a nucleobase deaminase enzyme and, in some cases, a DNA glycosylase inhibitor. Currently, there are 2 major categories of base editors, cytidine base editors (CBEs) and adenine base editors (ABEs), which catalyze C>T and A>G transitions. Base editors can be delivered, for example, via HDAd5/35++ vectors to efficiently edit promoters and enhancers to active or inactivate a gene. Exemplary methods are described in U.S. Pat. Nos. 9,840,699; 10,167,457; 10,113,163; 11,306,324; 11,268,082; 11,319,532; and 11,155,803. Also contemplated are prime editors that comprise a reverse transcriptase conjugated to (e.g., fused with) a Cas endonuclease and a polynucleotide useful as a DNA synthesis template conjugated to (e.g., fused with) a guide RNA, as described in WO 2020/191153.

Exemplary vectors that can be used for the genome editing applications include, but are not limited to, plasmids, retroviral vectors, lentiviral vectors, adenovirus vectors (e.g., Ad5/35, Ad5, Ad26, Ad34, Ad35, Ad48, parvovirus (e.g., adeno-associated virus (AAV) vectors, herpes simplex virus vectors, baculoviral vectors, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including herpes virus (e.g., Herpes Simplex virus types 1 and 2, Epstein-Barr virus, cytomegalovirus), and poxvirus (e.g., canarypox, vaccinia or modified vaccinia virus. The vector comprising the nucleic acid molecule of interest may be delivered to the cell (e.g., iPS cells, endothelial cells, hemogenic endothelial cells, HSCs (ST-HSCs or LT-HSCs) via any method known in the art, including but not limited to transduction, transfection, infection, and electroporation. Any of these vectors may include transposable element (such as a piggyback transposon or sleeping beauty transposon). Transposons insert specific sequences of DNA into genomes of vertebrate animals. The gene of interest can be integrated into the genome of a mammalian cell by transposase-catalyzed cleavage of similar excision sites that exist within the nuclear genome of the cell.

For increased efficiency, in some embodiments, the Cas and the gRNA can be combined before being delivered into the cells. The Cas-gRNA complex is known as a ribonucleoprotein (RNP). A number of methods have been developed for direct delivery of RNPs to cells. For example, RNP can be delivered into cells in culture by lipofection or electroporation. Electroporation using a nucleofection protocol can be employed, and this procedure allows the RNP to enter the nucleus of cells quickly, so it can immediately start cutting the genome. See, for example, Zhang S, Shen J, Li D, Cheng Y. Strategies in the delivery of Cas9 ribonucleoprotein for CRISPR/Cas9 genome editing. Theranostics. 2021 Jan. 1; 11 (2): 614-648, hereby incorporated by reference in its entirety. In some embodiments, Cas9 and gRNA are electroporated as RNP into the donor iPSCs and/or HSCs.

Generally, a protospacer adjacent motif (PAM) is required for a Cas nuclease to cut and is generally found 3-4 nucleotides downstream from the cut site. The PAM is a short DNA sequence (usually 2-6 base pairs in length) that follows the DNA region targeted for cleavage by the CRISPR system, such as CRISPR-Cas9. In some embodiments, the PAM sequences, sgRNAs, or base editing tools targeting haplotypes or polymorphs of HLA loci does not include four Gs, four Cs, GC repeats, or combinations thereof.

In some embodiments, a CRISPR/Cas9 system specific to a unique HLA haplotype can be developed by designing singular gRNAs targeting each of the donor-specific HLA-A, HLA-DPB1, and HLA-DQB1 genes (for example), using the gRNAs as described herein. To perform genetic knockout, the gRNA targets the Cas9 protein to the appropriate site to edit. Next, the Cas9 protein can perform a double strand break (DSB), where the DNA repairs through a non-homologous end joining (NHEJ) mechanism which generates indels resulting in a frameshift mutation and terminates the resulting protein's function. However, off-target genetic modifications can occur and alter the function of otherwise intact genes. For example, the Cas9 endonuclease can create DSBs at undesired off-target locations, even in the presence of some degree of mismatch. This off-target activity can create genome instability events, such as point mutations and genomic structural variations. In various embodiments, a sgRNA targeting HLA-A can target a region of chromosome 6 defined as 29942532-29942626. In various embodiments, a sgRNA targeting HLA-DQB1 can target a region of chromosome 6 defined as 32665067-32664798. In various embodiments, a sgRNA targeting HLA-DPB1 can target a region of chromosome 6 defined as 33080672-33080935.

gRNAs can be used to develop clonal iPSCs. Such iPSC lines can be evaluated for (i) ON-target edits, (ii) OFF-target edits, and (iii) Translocation edits, for example using sequencing, as described herein. Specifically, such assays can be performed by multiplex PCR with primers designed to target and enrich regions of interest followed by next-generation sequencing (e.g., Amplicon sequencing, AMP-seq). The ON-target panel and the translocation panel can amplify the intended edited region, allowing for selection of iPSC clones with the expected edits which are free from chromosomal translocation arising from unintended DSB cut-site fusion. The OFF-target panel can enrich any potential off-target regions identified via sequencing and allows for selection of iPSC clones with negligible off-target mutations. Together, these assays enable a screen of the iPSC clones to select the clones with the desired edits, while excluding potential CRISPR/Cas9-related genome integrity issues.

In some embodiments, to further ensure the genomic stability and integrity of reprogrammed and edited iPSCs, genetic and genomic assays can be performed to select for clones which, for example, did not undergo translocation and mutation events, and that did not integrate the episomal vectors. For example, whole-genome sequencing (WGS) is performed on CD34+ cells and on iPSC clones after reprogramming, where the genomes are compared for differences arising from editing. These analyses provide an assessment of which iPSC clone genomes differ from the CD34+ starting material, enabling informed selection iPSC clones which did not accrue mutations during the reprogramming.

In some embodiments, karyotyping analyses using systems such as KARYOSTAT assays is used to select iPSC clones which did not accrue indels and translocation during the reprogramming, for example as described in Ramme A P, et al, “Supporting dataset of two integration-free induced pluripotent stem cell lines from related human donors,” Data Brief. 2021 May 15; 37:107140, hereby incorporated by reference in its entirety. KARYOSTAT assays allow for visualization of chromosome aberrations with a resolution similar to G-banding karyotyping. The size of structural aberration that can be detected is >2 Mb for chromosomal gains and >1 Mb for chromosomal losses. The KARYOSTAT array is functionalized for balanced whole-genome coverage with a low-resolution DNA copy number analysis, where the assay covers all 36,000 RefSeq genes, including 14,000 OMIM targets. The assay enables the detection of aneuploidies, submicroscopic aberrations, and mosaic events.

In some embodiments, Array Comparative Genomic Hybridization (aCGH) analyses is used to select iPSC clones which did not accrue copy number aberrations (CNA) during reprogramming, for example as described in Wiesner et al. “Molecular Techniques,” Editor(s): Klaus J. Busam, Pedram Gerami, Richard A. Scolyer, “Pathology of Melanocytic Tumors,” Elsevier, 2019, pp. 364-373, ISBN 9780323374576; and Hussein S M, et al. “Copy number variation and selection during reprogramming to pluripotency,” Nature. 2011 Mar. 3; 471 (7336): 58-62, hereby incorporated by reference in its entirety. aCGH is a technique that analyzes the entire genome for CNA by comparing the sample DNA to reference DNA.

In some embodiments, targeted heme malignancy NGS panel analyses is used to select iPSC clones which did not accrue hematologic malignancy mutations during reprogramming. For example, targeted heme malignancy NGS panels can focus on myeloid leukemia, lymphoma, and/or other hematologic malignancy-associated genes to generate a smaller, more manageable data set than broader methods. Targeted heme malignancy NGS panel analysis includes the use of highly multiplexed PCR to amplify regions associated with hematologic malignancies followed by next-generation sequencing.

In some embodiments, Droplet Digital PCR (ddPCR) is used to select iPSC clones which did not integrate episomal vectors and that have been passaged enough for episomal vector clearance. As discussed herein, iPSC reprogramming of CD34+ cells can be achieved by delivering episomal vectors encoding reprogramming factors. However, episomal vectors can, albeit rarely, randomly integrate into the cellular genome, which could disrupt developmental processes, homeostasis, etc. Therefore, ddPCR methods can be used to detect residual episomal vector in the iPSC cultures and enable selection of iPSC clones which did not integrate episomal vectors.

In some embodiments, after assessing that the selected clones are free from genomic aberrations related to editing, the clones can be additionally tested for spontaneous mutations that might arise during expansion. For example, mutations affecting hematologic malignancy genes, indel, translocations, number aberrations, e.g., as described for the pre-edited reprogrammed clones. Analyses for spontaneous mutations can include whole-genome sequencing (WGS), KARYOSTAT analysis, Array Comparative Genomic Hybridization (aCGH) analysis, targeted heme malignancy NGS panel AMP-Seq analysis, and/or Droplet Digital PCR (ddPCR).

In certain embodiments, iPSCs are homozygous for at least one retained Class I and Class II loci. In some embodiments, iPSCs are derived from T cells, for example, with a known or unknown TCR specificity. In some embodiments, the T cells bear TCRs with specificity for one or more autoantigens or other antigen of interest. Exemplary autoantigens are described herein. In still other embodiments, the iPSCs can be gene edited to express a chimeric antigen receptor (CAR) to direct the resulting T cells to a tissue or organ of interest.

Somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral, Sendai, or measles viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations.

In some embodiments, the human pluripotent stem cells (e.g., iPSCs) are gene-edited. Gene-editing can include, but is not limited to, modification of HLA genes (e.g., deletion of one or more HLA Class I and/or Class II genes), deletion of β2 microglobulin (β2M), deletion of CIITA, deletion or addition of T Cell Receptor (TCR) genes, or addition of a chimeric antigen receptor (CAR) gene, for example. An exemplary CAR can target any desired organ or tissue specific antigen, and in some embodiments is specific for an antigen unique to a donor organ. For example, the iPSCs can be T-cell receptor (TCR)-transduced iPSCs. Exemplary TCRs can be specific for autoantigens of interest. Such embodiments enable the production of large-scale regenerated Tregs with a desired antigen-specificity. Alternatively, engineered iPSCs with one or more HLA knockouts and TCR knockouts can be placed in a bioreactor for a feeder-and-serum-free differentiation, under GMP-grade conditions, to generate fully functional and histocompatible T cells.

In some embodiments, iPSCs are prepared from CD3⁺ cells or in some embodiments T lymphocytes (T-iPSCs). For example, T lymphocytes can be isolated with a desired antigen specificity (using for example, cell sorting with HLA-peptide ligands), and reprogrammed to T-iPSCs. These T-iPSCs are then redifferentiated into a population comprising the desired T cell population or T-progenitor population according to this disclosure. When T-iPSCs are produced from antigen-specific T cells, T-iPSCs inherit the rearranged T cell receptor (TCR) genes. In these embodiments, T cells redifferentiated from the T-iPSCs demonstrate the same antigen specificity as the original T cells.

In various embodiments, iPSCs are prepared, and expanded using a culture system. Expanded iPSCs can be recovered from the culture for differentiating to embryoid bodies (EBs). EBs, created by differentiation of iPSCs, are three-dimensional aggregates of iPSCs and comprise the three (or alternatively two or one) embryonic germ layer(s) based on the differentiation method(s). Preparation of EBs is described, for example, in US 2019/0177695, which is hereby incorporated by reference in its entirety. In some embodiments, EBs prepared by differentiation of the iPSCs, are expanded in a bioreactor as described, for example, in Abecasis B. et al., Expansion of 3D human induced pluripotent stem cell aggregates in bioreactors: Bioprocess intensification and scaling-up approaches. J. of Biotechnol. 246 (2017) 81-93. EBs can be used to generate any desired cell type. Other methods, including a 3D suspension culture, for expansion or differentiation of EBs is described in WO 2020/086889, which is hereby incorporated by reference in its entirety.

In some embodiments, the process according to each aspect can comprise generating CD34+-enriched cells from the pluripotent stem cells (e.g., EBs) and inducing endothelial-to-hematopoietic differentiation. HSCs comprising relatively high frequency of LT-HSCs can be generated from the cell populations using various stimuli or factors, including mechanical, biochemical, metabolic, and/or topographical stimuli, as well as factors such as extracellular matrix, niche factors, cell-extrinsic factors, induction of cell-intrinsic properties; and including pharmacological and/or genetic means.

In some embodiments, the method comprises preparing endothelial cells with hemogenic potential from pluripotent stem cells, prior to induction of EHT. In some embodiments, the over-expression of GATA2/ETV2, GATA2/TAL1, or ER71/GATA2/SCL can lead to the formation of endothelial cells with hemogenic potential from PSC sources. In some embodiments, the method comprises overexpression of E26 transformation-specific variant 2 (ETV2) transcription factor in the iPSCs. Following CD34+ enrichment, HSCs are then generated from the endothelial cells using mechanical, biochemical, pharmacological and/or genetic stimulation or modification. ETV2 can be expressed by introduction of an encoding non-integrating episomal plasmid, for constitutive or inducible expression of ETV2, and for production of transgene-free hemogenic ECs. In some embodiments, ETV2 is expressed from an mRNA introduced into the iPSCs. mRNA can be introduced using any available method, including electroporation or lipofection. Differentiation of cells expressing ETV2 can comprise addition of VEGF-A. See, Wang K, et al., Robust differentiation of human pluripotent stem cells into endothelial cells via temporal modulation of ETV2 with mRNA. Sci. Adv. Vol. 6 (2020). Cells generated in this manner may be used for robust production of CD34+ cells followed by EHT induction according to embodiments of this disclosure.

In some embodiments, iPSC differentiation proceeds until cells are at least about 10% CD34+, or at least about 20% CD34+, or at least about 25% CD34+, or at least about 30% CD34+. In some embodiments, CD34+ enrichment and EHT may be induced at Day 8 to Day 14 of iPSC differentiation, such as for example, Day 8, Day 9, Day 10, Day 11, Day 12, Day 13, or Day 14. Differentiation of iPSCs can be according to known techniques. In some embodiments, iPSC differentiation involves factors such as, but not limited to, combinations of bFGF, Y27632, BMP4, VEGF, SCF, EPO, TPO, IL-6, IL-11, and/or IGF-1. In some embodiments, hPSCs are differentiated using feeder-free, serum-free, and/or GMP-compatible materials. In some embodiments, hPSCs are co-cultured with murine bone marrow-derived feeder cells such as OP9 or MS5 cell line in serum-containing medium. The culture can contain growth factors and cytokines to support differentiation of embryoid bodies or monolayer system. The OP9 co-culture system can be used to generate multipotent HSPCs, which can be differentiated further to several hematopoietic lineages including T lymphocytes. See Netsrithong R. et al., Multilineage differentiation potential of hematoendothelial progenitors derived from human induced pluripotent stem cells, Stem Cell Research & Therapy Vol. 11 Art. 481 (2020). Alternatively, a step-wise process using defined conditions with specific signals can be used. For example, the expression of HOXA9, ERG, RORA, SOX4, and MYB in human PSCs favors the direct differentiation into CD34+/CD45+ progenitors with multilineage potential. Further, expression of factors such as HOXB4, CDX4, SCL/TAL1, or RUNX1a support the hematopoietic program in human PSCs. See Doulatov S. et al., Induction of multipotential hematopoietic progenitors from human pluripotent stem cells via re-specification of lineage-restricted precursors, Cell Stem Cell. 2013 Oct. 3; 13 (4).

Induction of EHT can be with any known process. In some embodiments, induction of EHT generates an HSC population comprising LT-HSCs. In some embodiments, EHT generates a cell population comprising HSPCs. In some embodiments, EHT generates HSCs and/or HSPCs through endothelial or hemogenic endothelial cell (HEC) precursors using mechanical, biochemical, pharmacological and/or genetic means (e.g., via stimulation, inhibition, and/or genetic modifications). In some embodiments, the EHT generates a stem cell population comprising one or more of long-term hematopoietic stem cells (LT-HSCs), short-term hematopoietic stem cells (ST-HSCs), and HSPCs. In various embodiments, EHT can be induced in the culture for from 2 days to 12 days, such as about 4 days to about 8 days (e.g., about 4 days, about 5 days, about 6 days, about 7 days, or about 8 days). In some embodiments, EHT is induced in the culture from about 5 days to about 7 days.

In some embodiments, the HSC and/or HSPC population or fraction thereof is differentiated to T cells or progenitors or derivatives thereof independent of the use of an agonist of a mechanosensitive receptor or a mechanosensitive channel, such as Yoda1. In some embodiments, the use of an agonist of a mechanosensitive receptor or a mechanosensitive channel (e.g., Yoda1) is optional. Thus, in some embodiments, CD34+ cells are enriched from a differentiated pluripotent stem cell population to prepare a CD34+-enriched population. Endothelial-to-hematopoietic transition of the CD34+-enriched cell population is induced for at least two days, but no more than 12 days in which the use of an agonist of a mechanosensitive receptor or a mechanosensitive channel such as Yoda1, jedi1, jedi2, or ssRNA40 is optional. The HSCs and/or HSPCs are differentiated to a progenitor T cell population or a T cell population (e.g., as described herein). In some embodiments, the endothelial-to-hematopoietic transition of the CD34+-enriched cell population is induced for at least for two days, and further for about 4 hours, or about 8 hours, or about 12 hours, or about 16 hours, or about 20 hours, or about 24 hours, or about 2 days, or about 3 days, or about 4 days, or about 5 days, or about 6 days, or about 7 days, or about 8 days, or about 9 days, or about 10 days. The total EHT differentiation proceeds for no more than 12 days.

In some embodiments, the method comprises increasing the expression or activity of dnmt3b in PSCs, embryoid bodies, CD34+ cells, ECs, HECs or HSCs, which can be by mechanical, genetic, biochemical, or pharmacological means. In some embodiments, the method comprises increasing activity or expression of DNA (cytosine-5-)-methyltransferase 3 beta (Dnmt3b) and/or GTPase IMAP Family Member 6 (Gimap6) in the cells. See WO 2019/236943 and WO 2021/119061, which are hereby incorporated by reference in their entireties. In some embodiments, the induction of EHT comprises increasing the expression or activity of dnmt3b.

In some embodiments, cells are contacted with an effective amount of an agonist of a mechanosensitive receptor or a mechanosensitive channel that increases the activity or expression of Dnmt3b. In some embodiments, the mechanosensitive receptor is Piezol. An exemplary Piezol agonist is Yoda1. In some embodiments, the mechanosensitive receptor is Trpv4. An exemplary Trpv4 agonist is GSK1016790A. Yoda1 (2-[5-[[(2,6-Dichlorophenyl)methyl]thio]-1,3,4-thiadiazol-2-yl]-pyrazine) is a small molecule agonist developed for the mechanosensitive ion channel Piezol. Syeda R, Chemical activation of the mechanotransduction channel Piezol. eLife (2015). Yoda 1 has the following structure:

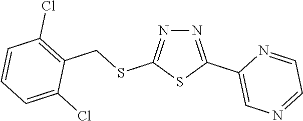 [SMILES: Clc1cccc(Cl)c1CSc1nnc(-c2cnccn2)s1]

Derivatives of Yoda1 can be employed in various embodiments. For example, derivatives comprising a 2,6-dichlorophenyl core are employed in some embodiments. Exemplary agonists are disclosed in Evans E L, et al., Yoda1 analogue (Dooku1) which antagonizes Yoda1-evoked activation of Piezo1 and aortic relaxation, British J. of Pharmacology 175 (1744-1759): 2018. Still other Piezol agonist include Jedi1, Jedi2, single-stranded (ss) RNA (e.g., ssRNA40) and derivatives and analogues thereof. See Wang Y., et al., A lever-like transduction pathway for long-distance chemical-and mechano-gating of the mechanosensitive Piezo1 channel. Nature Communications (2018) 9:1300; Sugisawa, et al., RNA Sensing by Gut Piezo1 Is Essential for Systemic Serotonin Synthesis, Cell, Volume 182, Issue 3, 2020, Pages 609-624, which are hereby incorporated by reference in their entireties. These Piezo1 agonists are commercially available. In various embodiments, the effective amount of the Piezo1 agonist or derivative is in the range of about 1 μM to about 500 μM, or about 5 μM to about 200 μM, or about 5 μM to about 100 μM, or in some embodiments, in the range of about 25 μM to about 150 μM, or about 25 μM to about 100 μM, or about 25 μM to about 50 μM. Alternatively, single-stranded (ss) RNA (e.g., ssRNA40), and derivatives and analogues thereof, can be used for Piezo1 activation.

In various embodiments, pharmacological Piezo1 activation is applied to CD34+ cells (i.e., CD34+-enriched cells). In certain embodiments, pharmacological Piezo1 activation may further be applied to iPSCs, embryoid bodies, ECs, hemogenic endothelial cells (HECs), HSCs, hematopoietic progenitors, as well as hematopoietic lineage(s). In certain embodiments, Piezo1 activation is applied at least to EBs generated from iPSCs, CD34+ cells isolated from EBs, and/or combinations thereof, which in accordance with various embodiments, allows for superior generation of T progenitor cells and T cell lineages derived therefrom (e.g., Tregs as well as mature «/β T cells) as compared to other methods for inducing EHT. Advantageously, progenitor T cells generated by Piezo1 activation during EHT have greater engraftment potential, then progenitor T cells prepared without Piezo1 activation during EHT.

In some embodiments, pharmacological agents, such as but not limited to retinoic acid, retinoid acid receptor (RAR) agonist, dibutyl cyclic AMP, protein kinase inhibitors, ascorbic acid, dexamethasone, forskolin (FSK), baicalin, or 2-methyl-5-hydroxytryptamine or a combination thereof is applied to CD34+ cells (i.e., CD34+-enriched cells). In certain embodiments, pharmacological agent activation may further be applied to iPSCs, embryoid bodies, ECs, hemogenic endothelial cells (HECs), HSCs, hematopoietic progenitors, as well as hematopoietic lineage(s) (e.g., to expand cell populations). In certain embodiments, agents, such as, retinoic acid or a retinoid acid receptor (RAR) agonist activation is applied at least to EBs generated from iPSCs, CD34+ cells isolated from EBs, and/or combinations thereof.

Alternatively or in addition, the activity or expression of Dnmt3b can be increased directly in the cells, e.g., in CD34+-enriched cells. For example, mRNA expression of Dnmt3b can be increased by delivering Dnmt3b-encoding transcripts to the cells, or by introducing a Dnmt3b-encoding transgene, or a transgene-free method, not limited to introducing a non-integrating episome to the cells. In some embodiments, gene editing is employed to introduce a genetic modification to Dnmt3b expression elements in the cells, such as, but not limited to, to increase promoter strength, ribosome binding, RNA stability, and/or impact RNA splicing.

In some embodiments, the method comprises increasing the activity or expression of Gimap6 in the cells, alone or in combination with Dnmt3b and/or other genes that are up- or down regulated upon cyclic strain or Piezol activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene. In some embodiments, gene editing is employed to introduce a genetic modification to Gimap6 expression elements in the cells (such as one or more modifications to increase promoter strength, ribosome binding, RNA stability, or to impact RNA splicing).

In embodiments of this disclosure employing mRNA delivery to cells, known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response.

In some embodiments, expression of Dnmt3b and/or Gimap6 is increased by introducing a transgene into the cells, which can direct a desired level of overexpression (with various promoter strengths or other selection of expression control elements). Transgenes can be introduced using various viral vectors or transfection reagents (including Lipid Nanoparticles) as are known in the art. In some embodiments, expression of Dnmt3b and/or Gimap6 is increased by a transgene-free method (e.g., episome delivery). In some embodiments, expression or activity of Dnmt3b and/or Gimap6 or other genes disclosed herein are increased using a gene editing technology, for example, to introduce one or more modifications to increase promoter strength, ribosome binding, or RNA stability.

In some embodiments, the method comprises applying cyclic 2D, 3D, or 4D stretch to cells. In various embodiments, the cells subjected to cyclic 2D, 3D, or 4D stretch are selected from one or more of CD34+-enriched cells, iPSCs, ECs, and HECs. For example, a cell population is introduced to a bioreactor that provides a cyclic-strain biomechanical stretching, as described in WO 2017/096215, which is hereby incorporated by reference in its entirety. The cyclic-strain biomechanical stretching can increase the activity or expression of Dnmt3b and/or Gimap6. In these embodiments, mechanical means apply stretching forces to the cells, or to a cell culture surface having the cells (e.g., ECs or HECs) cultured thereon. For example, a computer controlled vacuum pump system or other means for providing a stretching force (e.g., the FlexCell™ Tension System, the Cytostretcher System) attached to flexible biocompatible and/or biomimetic surface can be used to apply cyclic 2D, 3D, or 4D stretch ex vivo to cells under defined and controlled cyclic strain conditions. For example, the applied cyclic stretch can be from about 1% to about 20% cyclic strain (e.g., about 6% cyclic strain) for several hours or days (e.g., about 7 days). In various embodiments, cyclic strain is applied for at least about one hour, at least about two hours, at least about six hours, at least about eight hours, at least about 12 hours, at least about 24 hours, at least about 48 hours, at least about 72 hours, at least about 96 hours, at least about 120 hours, at least about 144 hours, or at least about 168 hrs.

Alternatively or in addition, EHT is stimulated by Trpv4 activation. The Trpv4 activation can be by contacting cells (e.g., CD34+-enriched cells, ECs, or HECs) with one or more Trpv4 agonists, which are optionally selected from GSK1016790A, 4alpha-PDD, or analogues and/or derivatives thereof.

Where cell populations are described herein as having a certain phenotype it is understood that the phenotype represents a significant portion of the cell population, such as at least 25%, at least 40%, or at least about 50%, or at least about 60%, or at least about 75%, or at least about 80%, or at least about 90% of the cell population. Further, at various steps, cell populations can be enriched for cells of a desired phenotype, and/or depleted of cells of an undesired phenotype, such that cell population comprise at least about 75%, or at least about 80%, or at least about 90% of the desired phenotype. Such positive and negative selection methods are known in the art. For example, cells can be sorted based on cell surface antigens (including those described herein) using a fluorescence activated cell sorter, or magnetic beads which bind cells with certain cell surface antigens. Negative selection columns can be used to remove cells expressing undesired cell-surface markers. In some embodiments, cells are enriched for CD34+ cells (prior to and/or after undergoing EHT). In some embodiments, the cell population is cultured under conditions that promote expansion of CD34+ cells to thereby produce an expanded population of stem cells. In various embodiments, cells are enriched for T progenitor cells (using cell surface markers described herein, such as CD7+), and which may be optionally differentiated further in culture. Further, after progenitor T cell or T cell differentiation, cells may be enriched for markers such as CD3, CD4, and/or CD8, for example. Tregs can further be enriched in the resulting population by CD25+ enrichment, for example.

In various embodiments, CD34+ cells (e.g., the floater and/or adherent cells) are harvested from the culture undergoing endothelial-to-hematopoietic transition between Day 10 to Day 20 of iPSC differentiation, such as from Day 10 to Day 17, or from Day 12 to Day 15 of iPSC differentiation.

In various embodiments, the HSC and/or HSPC population (e.g., or CD34+-enriched cells isolated therefrom) are further expanded. For example, the cells can be expanded according to methods disclosed in U.S. Pat. Nos. 8,168,428; 9,028,811; 10,272,110; and 10,278,990, which are hereby incorporated by reference in their entireties. In some embodiments, ex vivo expansion of HSCs or CD34+-enriched cells employs prostaglandin E₂ (PGE2) or a PGE₂ derivative. In some embodiments of this disclosure, the HSCs comprise at least about 0.01% LT-HSCs, or at least about 0.05% LT-HSCs, or at least about 0.1% LT-HSCs, or at least about 0.5% LT-HSCs, or at least about 1% LT-HSCs.

Hematopoietic stem cells (HSCs) which give rise to erythroid, myeloid, and lymphoid lineages, can be identified based on the expression of CD34 and the absence of lineage specific markers (termed Lin−). In some embodiments, a population of stem cells comprising HSCs are enriched, for example, as described in U.S. Pat. No. 9,834,754, which is hereby incorporated by reference in its entirety. For example, this process can comprise sorting a cell population based on expression of one or more of CD34, CD90, CD38, and CD43. A fraction can be selected for further differentiation that is one or more of CD34⁺, CD90⁺, CD38⁻, and CD43⁻. In some embodiments, the stem cell population for differentiation to a hematopoietic lineage is at least about 80% CD34⁻, or at least about 90% CD34⁺, or at least about 95% CD34⁺.

In some embodiments, the HSC/HSPC population, or CD34+-enriched cells or fraction thereof, or derivative population are expanded as described in US 2020/0308540, which is hereby incorporated by reference in its entirety. For example, the cells are expanded by exposing the cells to an aryl hydrocarbon receptor antagonist including, for example, SR1 or an SR1-derivative. See also, Wagner et al., Cell Stem Cell 2016; 18 (1): 144-55 and Boitano A., et al., Aryl Hydrocarbon Receptor Antagonists Promote the Expansion of Human Hematopoietic Stem Cells. Science 2010 Sep. 10; 329 (5997): 1345-1348.

In some embodiments, the compound that promotes expansion of CD34+ cells includes a pyrimidoindole derivative including, for example, UM171 or UM729 (see US 2020/0308540, which is hereby incorporated by reference).

In some embodiments, the stem cell population or CD34+-enriched cells are further enriched for cells that express Periostin and/or Platelet Derived Growth Factor Receptor Alpha (pdgfra) or are modified to express Periostin and/or pdgfra, as described in WO 2020/205969 (which is hereby incorporated by reference in its entirety). Such expression can be by delivering encoding transcripts to the cells, or by introducing an encoding transgene, or a transgene-free method, not limited to introducing a non-integrating episome to the cells. In some embodiments, gene editing is employed to introduce a genetic modification to expression elements in the cells, such as to modify promoter activity or strength, ribosome binding, RNA stability, or impact RNA splicing.

In still other embodiments, the stem cell population or CD34+-enriched cells are cultured with an inhibitor of histone methyltransferase EZH1. Alternatively, EZH1 is partially or completely deleted or inactivated or is transiently silenced in the stem cell population (e.g., by siRNA). Inhibition of EZH1 can direct myeloid progenitor cells (e.g., CD34+CD45+) to lymphoid lineages. See WO 2018/048828, which is hereby incorporated by reference in its entirety. In still other embodiments, EZH1 is overexpressed in the stem cell population.

In various embodiments, the population comprising HSCs and/or HSPCs or fraction thereof is differentiated to a population comprising T progenitor cells or T lymphocytes.

In some embodiments, the cell population is cultured with a Notch ligand, partial or full, SHH, extracellular matrix component(s), and/or combinations thereof, ex vivo, to differentiate HSCs to T cell population (or precursor thereof). Further, according to known processes, xenogenic OP9-DL1 cells are often employed for differentiation to T cells. The OP9-DL1 co-culture system uses a bone marrow stromal cell line (OP9) transduced with the Notch ligand delta-like-1 (DLL1) to support T cell development from stem cell sources. The OP9-DL1 system limits the potential of the cells for clinical application. There is a need for feeder-cell-free systems that can generate T lymphocytes from hiPSCs for clinical use, and in some embodiments the present invention meets this objective.

The term “Notch ligand” as used herein refers to a ligand capable of binding to a Notch receptor polypeptide present in the membrane of a hematopoietic stem cell or progenitor T cell. The Notch receptors include Notch-1, Notch-2, Notch-3, and Notch-4. Notch ligands typically have a DSL domain (D-Delta, S-Serrate, and L-Lag2) comprising 20-22 amino acids at the amino terminus, and from 3 to 8 EGF repeats on the extracellular surface. In various embodiments, the Notch ligand comprises at least one of Delta-Like-1 (DLL1), Delta-Like-4 (DLL4), SFIP3, DeltaMax (disclosed in PCT/US2020/041765 and PCT/US2020/030977, which are incorporated herein in their entirety by reference) or a functional portion thereof. A key signal that is delivered to incoming lymphocyte progenitors by the thymus stromal cells in vivo is mediated by DL4, which is expressed by cortical thymic epithelial cells.

The earliest intrathymic progenitors express high levels of CD34 and CD7, do not express CD1a, and are triple-negative (TN) for mature T cell markers: CD4, CD8, and CD3. Commitment to the T cell lineage is associated with the expression of CD1a by CD7-expressing pro-thymocytes. Thus, immature stages of T-cell development are typically delineated as CD34⁺CD1a⁻ (most immature) and CD34⁺CD1a⁺ cells. The transition from CD34⁺CD7⁺CD1a⁻ to CD34⁺CD7⁺CD1a⁺ by early thymocytes is associated with T-cell commitment. CD34⁺CD7⁺CD1a⁺ cells are likely T-lineage restricted. Following this stage, thymocytes progress to a CD4 immature single positive stage, at which point CD4 is expressed in the absence of CD8. Thereafter, a subset of the cells differentiates to the CD4⁺CD8⁺ double positive (DP) stage. Finally, following TCRα rearrangement, TCRαβ-expressing DP thymocytes undergo positive and negative selection, and yield CD4⁺CD8⁻ and CD4⁻CD8⁺ single positive (SP) T-cells.

In some embodiments, progenitor T cells are isolated by enrichment for CD7 expression. In some embodiments, progenitor T cells are expanded as described in US 2020/0308540, which is hereby incorporated by reference in its entirety. For example, the cells may be expanded by exposing the cells to an aryl hydrocarbon receptor antagonist including, for example, SR1 or an SR1-derivative. See also, Wagner et al., Cell Stem Cell 2016; 18(1): 144-55. In some embodiments, the compound that promotes expansion includes a pyrimidoindole derivative including, for example, UM171 or UM729 (see US 2020/0308540, which is hereby incorporated by reference).

Differentiation to progenitor T cells can further include in some embodiments the presence of stem cell factor (SCF), Flt3L and interleukin (IL)-7. In various embodiments, CD7+ progenitor T cells created express CD1a. The CD7+ progenitor T cells do not express CD34 or express a diminished level of CD34 compared to the HSC population. In some embodiments, the CD7+ progenitor T cells (or a portion thereof) further express CD5. Accordingly, the phenotype of the progenitor T cells may comprise CD7⁺CD1a⁺ cells. In some embodiments, the phenotype of the progenitor T cells comprises CD7⁺CD5⁺ cells. In some embodiments, the progenitor T cells comprise CD7⁺CD1a⁺CD5⁺ cells, and optionally CD34+.

In some embodiments, the progenitor T cells exhibit a diminished level of CD34 expression, minimal CD34 expression (compared to the HSC population), or no CD34 expression. In some embodiments, CD34 expression is diminished in the population by at least about 50%, or at least about 75%, relative to the HSC population.

In some embodiments, the Notch ligand is an anti-Notch (agonistic) antibody that can bind and engage Notch signaling. In some embodiments, the antibody is a monoclonal antibody (including a human or humanized antibody), a single chain antibody (scFv), a nanobody, or other antibody fragment or antigen-binding molecule capable of activating the Notch signaling pathway.

In some embodiments, the Notch ligand is a Delta family Notch ligand. The Delta family ligand in some embodiments is Delta-1 (Genbank Accession No. AF003522, Homo sapiens), Delta-like 1 (DLL1, Genbank Accession No. NM_005618 and NP_005609, Homo sapiens; Genbank Accession No. X80903, 148324, M. musculus), Delta-4 (Genbank Accession No. AF273454, BAB18580, Mus musculus; Genbank Accession No. AF279305, AAF81912, Homo sapiens), and/or Delta-like 4 (DLL4; Genbank Accession. No. Q9NR61, AAF76427, AF253468, NM_019074, Homo sapiens; Genbank Accession No. NM 019454, Mus musculus). Notch ligands are commercially available or can be produced, for example, by recombinant DNA techniques.

In some embodiments, the Notch ligand comprises an amino acid sequence that is at least about 70%, or at least about 80%, or at least about 90%, or at least about 95%, or at least about 97% identical (e.g., about 100% identical) to human DLL1 or DLL4 Notch ligand. Functional derivatives of Notch ligands (including fragments or portions thereof) will be capable of binding to and activating a Notch receptor. Binding to a Notch receptor may be determined by a variety of methods known in the art including in vitro binding assays and receptor activation/cell signaling assays.

In some embodiments, the Notch ligand is a DLL4 having one or more affinity enhancing mutations, such as one or more (or all) of: G28S, F107L, 1143F, H194Y, L206P, N257P, T271L, F280Y, S301R and Q305P, with respect to hDLL4. See Gonzalez-Perez, et al., Affinity-matured DLL4 ligands as broad-spectrum modulators of Notch signaling, Nature Chemical Biology (2022).

In various embodiments, the Notch ligands are soluble, and are optionally immobilized on microparticles or nanoparticles, which are optionally paramagnetic to allow for magnetic enrichment or concentration processes. In still other embodiments, the Notch ligands are immobilized on a 2D or 3D culture surface, optionally with other adhesion molecules such as VCAM-1. See US 2020/0399599, which is hereby incorporated by reference in its entirety. In other embodiments, the beads or particles are polymeric (e.g., polystyrene or PLGA), gold, iron dextran, or constructed of biological materials, such as particles formed from lipids and/or proteins. In various embodiments, the particle has a diameter or largest dimension of from about 0.01 μm (10 nm) to about 500 μm (e.g., from about 1 μm to about 7 μm). In still other embodiments, polymeric scaffolds with conjugated ligands can be employed, as described in WO 2020/131582, which is hereby incorporated by reference in its entirety. For example, scaffold can be constructed of polylactic acid, polyglycolic acid, PLGA, alginate or an alginate derivative, gelatin, collagen, agarose, hyaluronic acid, poly(lysine), polyhydroxybutyrate, poly-epsilon-caprolactone, polyphosphazines, poly(vinyl alcohol), poly(alkylene oxide), poly(ethylene oxide), poly(allylamine), poly(acrylate), poly(4-aminomethylstyrene), pluronic polyol, polyoxamer, poly(uronic acid), poly(anhydride), poly(vinylpyrrolidone), and any combination thereof. In some embodiments, the scaffold comprises pores having a diameter between about 1 μm and 100 μm.

In some embodiments, the C-terminus of the Notch ligand is conjugated to the selected support. In some embodiments, this can include adding a sequence at the C-terminal end of the Notch ligand that can be enzymatically conjugated to the support, for example, through a biotin molecule. In another embodiment, a Notch ligand-Fc fusion is prepared, such that the Fc segment can be immobilized by binding to protein A or protein G that is conjugated to the support. Of course, any of the known protein conjugation methods can be employed.

Thus, in various embodiments, the Notch ligand is immobilized, functionalized, and/or embedded in 2D or 3D culture system. The Notch ligand may be incorporated along with a component of extracellular matrix, such as one or more selected from fibronectin, RetroNectin, and laminin. In some embodiments, the Notch ligand and/or component of extracellular matrix are embedded in inert materials providing 3D culture conditions. Exemplary materials include, but are not limited to, cellulose, alginate, and combinations thereof. In some embodiments, the Notch ligand, a component of extracellular matrix, or combinations thereof, are in contact with culture conditions providing topographical patterns and/or textures (e.g., roughness) to cells conducive to differentiation and/or expansion.

In some embodiments, HSCs are differentiated to progenitor T cells by culture in medium comprising TNF-α and/or antagonist of aryl hydrocarbon/dioxin receptor (SR1), and in the presence of Notch ligand. See US 2020/0390817, US 2021/0169934, and US 2021/0169935, which are hereby incorporated by reference in its entirety. In some embodiments the HSCs are cultured in a medium comprising TNF-α, IL-7, thrombopoietin (TPO), Flt3L, and stem cell factor (SCF), and optionally SR1, in the presence of an immobilized Delta-Like-4 ligand and a fibronectin fragment. In some embodiments, the cells are cultured with RetroNectin, which is a recombinant human fibronectin containing three functional domains: the human fibronectin cell-binding domain (C-domain), heparin-binding domain (H-domain), and CS-1 sequence domain. In some embodiments, cells are cultured in the presence of an immobilized Delta-Like-4 ligand and a RetroNectin. In some embodiments, cells are cultured in the presence of an immobilized Delta-Like-4 ligand, TNF-alpha, and a RetroNectin. In some embodiments, cells are cultured in the presence of an immobilized Delta-Like-1 ligand and a RetroNectin. In some embodiments, cells are cultured in the presence of SFIP3 and RetroNectin. In some embodiments, cells are cultured in the presence of an immobilized Delta-Like-4 ligand and SHH molecules and/or functional derivatives thereof. Exemplary fibronectin fragments include one or more RGDS, CS-1, and heparin-binding motifs. Fibronectin fragments can be free in solution or immobilized to the culture surface or on particles. In some embodiments, cells are cultured for 5 to 7 days to prepare CD7+ progenitor T cells. In some embodiments, cells are cultured for 8 to 13 days to prepare pre-T cells (e.g., CD34−, CD7+, CD5+/−). In some embodiments, cells are cultured for 15 to 21 days for production of mature T cells (e.g., CD3+). In some embodiments, cells are cultured for 21 days or more for production of Tregs.

In various embodiments, the method produces a Treg population by culturing the population comprising HSCs and/or HSPCs with the Notch ligand (including any of the embodiments described above) with or without component(s) extracellular matrix, and optionally adding TNF-alpha to the culture at certain stages of differentiation. Thus, cells created in some embodiments are progenitor or precursor cells committed to the T cell lineage (“progenitor T cells”). In some embodiments, the cells are CD7+ progenitor T cells. In some embodiments, the cells are CD25+ immature T cells, or cells that have undergone CD4 or CD8 lineage commitment. In some embodiments, the cells are CD4+CD8+ double positive (DP) or CD4+CD8− single positive cells. In some embodiments, the cells are single positive (SP) cells that are CD4+CD8−, and in some embodiments may be TCRhi. In some embodiments, the cells are TCRαβ+. In various embodiments, the cells are CD3+.

In various embodiments, the progenitor T cells are further cultured under suitable conditions to generate cells of a desired T cell population (e.g., Tregs), including with one or more Notch ligands. For example, the cells can be cultured in the presence of one or more Notch ligands as described for a sufficient time to form cells of the desired T cell population. In some embodiments, HSCs/HSPCs or progenitor T cells are cultured in suspension with soluble Notch ligand or Notch ligand conjugated to particles or other supports, or Notch ligand expressing cells. In some embodiments, the progenitor T cells or HSC/HSPC population are cultured in suspension or in adherent format in a bioreactor, optionally a closed or a closed, automated bioreactor, with a soluble or conjugated Notch ligand in suspension. One or more cytokines, extracellular matrix component(s), and thymic niche factor(s) that promote commitment and differentiation to the desired T cell population may also be added to the culture or reactor. In various embodiments, the HSC and/or HSPC population is cultured with the Notch ligand for about 4 to about 21 days, or from about 6 to about 18 days, or from about 7 to about 14 days to generate progenitor T cells. In some embodiments, the stem cell population or derivative thereof is cultured for at least about 21 days or at least about 28 days to generate the Treg lineage. In some embodiments, the stem cell population is cultured for less than about 28 days, or less than about 21 days, or less than about 15 days to produce the Treg population.

In various embodiments, the HSC/HSPC population is cultured in an artificial thymic organoid (ATO). See, Hagen, M. et al. (2019). The ATO will include culture of HSCs (or aggregates of HSCs) with a Notch ligand-expressing stromal cell line in serum-free conditions. The artificial thymic organoid is a 3D system, inducing differentiation of hematopoietic precursors to naive CD3+CD8+ and CD3+CD4+ T cells. In some embodiments, an artificial thymic organoid comprises DLL4 and BMP2, or functional fragments thereof.

In various embodiments, the method comprises generating Tregs. In certain embodiments, the Tregs express CD3 and a T cell receptor. In some embodiments, the Tregs comprise CD4⁺ cells, which are optionally expanded in culture. In some embodiments, the iPSCs, CD34+ cells, or derivatives thereof are modified to express a chimeric antigen receptor (CAR).

Regulatory T cells (Tregs) have the potential to be useful in a wide variety of clinical applications. For instance, they could be used to control the harmful immune responses seen in patients with autoimmune diseases such as childhood (Type I) diabetes, rheumatoid arthritis, multiple sclerosis, and inflammatory bowel disease; and to suppress rejection of transplanted organs in patients given heart, liver, or kidney transplants. However, due to a low frequency of Treg in peripheral blood (~1-2% in humans), its clinical application is limited. Accordingly, successful generation of Tregs from iPSCs ex vivo would fill a great need.

T lymphocytes and progenitor T cells can be differentiated to Tregs by expression of FOXP3. Tregs can optionally be further isolated or enriched by positive and/or negative selection. In various embodiments, this disclosure provides cell populations that comprise at least about 40%, or at least about 50%, or at least about 75%, or at least about 80%, or at least about 85%, or at least about 90% Tregs, and which may be antigen- or tissue-specific. Tregs can be defined as CD3+CD4+CD25+ and FoxP3+ cells. Additional cell surface markers in some embodiments include CTLA-4, CD39, CD73, GITR, and/or LAG-3. In exemplary embodiments, at least about 50%, or at least about 75%, or at least about 80% of the Tregs in the population express CTLA-4. In various embodiments, at least at least about 50%, or at least about 75%, or at least about 80% of the Tregs in the population express CD39 and/or CD73.

In some embodiments, pluripotent stem cells are cultured under conditions allowing for formation of embryoid bodies. Embryoid bodies are dissociated and CD34+ cells are isolated and used for induction of EHT, followed by differentiation to T cells or progenitor T cells by culturing the cells undergoing EHT with at least one Notch ligand (as described further herein). A vector is introduced during or subsequent to T cell differentiation that comprises a nucleic acid sequence encoding Foxp3, thereby driving formation of Tregs. In some embodiments, the population of T cells also expresses an exogenous gene to provide a tissue targeting function, such as a tissue specific T-cell receptor (TCR). For example, engineered Tregs are also engineered to express a pancreatic islet-specific T cell receptor (TCR) to target the engineered Tregs to the site of pancreatic-related disease. Optionally, the engineered Tregs include an insertion of an IL-2 signaling complex, which provides the engineered Tregs with proliferative and functional advantage.

Tregs can be defined as CD4⁺CD25⁺. Tregs control the immune response to self and foreign antigens and help prevent autoimmune disease. Differentiation of cells to Tregs in some embodiments involves modifying Treg precursors (e.g., CD4⁺αβ T cells or precursors thereof) to constitutively express FOXP3. The FOXP3 gene provides instructions for producing the forkhead box P3 (FOXP3) protein. The FOXP3 protein is a transcription factor involved in regulating the immune system, and is involved in the production of regulatory T cells. In some embodiments, the iPSCs, the CD34+ cells (e.g., isolated before or after EHT), progenitor T cells, CD25+ T cells, CD4+CD8+ cells, or CD4+ cells (e.g., αβ T cells) are gene edited to provide for expression of FOXP3, which can be constitutive and stable expression. In some embodiments, a regulatory sequence comprising a strong enhancer and/or promoter are inserted to operably control the expression of the FOXP3 gene in a constitutive and stable manner. In some embodiments, an enhancer binding domain is placed upstream of the FOXP3 promoter to activate the promoter to increase transcription of the FOXP3 gene. In some embodiments, a transcriptional activation domain is inserted, which includes specific DNA sequences that can be bound by a transcription factor, in which the transcription factor can thereby control the rate of transcription. Specific transcription factors can include but are not limited to SP1, AP1, C/EBP, heat shock factor, ATF/CREB, c-Myc, Oct-1 and/or NF-1. In some embodiments, the activator domains are used to silence the inhibition mechanisms that prevent transcription of the FOXP3 gene. In some embodiments, a FOXP3 gene (comprising coding sequence with constitutive expression control sequences) are inserted to provide for FOXP3 constitutive expression. Various manners of introducing donor templates, gene editing proteins, and gRNA are known, including the use of viral vectors such as AAV and lipid nanoparticles. See US 2021/0253652, which is hereby incorporated by reference in its entirety. In some embodiments, a FOXP3 donor gene with constitutive expression control sequences is inserted using CRISPR/Cas9.

In some embodiments, Tregs are expanded in culture in the presence of growth factors, such as IL-2. In addition, expansion protocols may include use of anti-CD3 and agonistic anti-CD28 antibodies, which can be conjugated to substrate surface (including beads) or provided in soluble form. In some embodiments, expansion of Tregs is not desired (or expansion is minimal), avoiding the loss of desired function through further culturing. Further, Tregs may have limited proliferative capacity. In some embodiments, Tregs are expanded in culture for 7 days or less, or about 4 days or less, or 2 days or less. In some embodiments, the Tregs are engineered to have a proliferative advantage, for example, by expressing a signaling complex, as described for example, in US 2021/0253652, which is hereby incorporated by reference in its entirety. In some embodiments, the signaling complex involves interleukin-2 receptor components, and optionally involves receptor signaling subunits that are shared by IL-2 and IL-15. In some embodiments, the signaling complex is as described in US 2020/0123224, the disclosure of which is hereby incorporated by reference in its entirety. For example, each chimeric protein component of the complex may have one half of a rapamycin binding complex as an extracellular domain, fused to one half of an intracellular signaling complex (e.g., an IL-2 signaling complex). Delivery of nucleic acids encoding the signaling complex to host cells permits intracellular signaling in the cells that can be controlled by the presence of rapamycin or a rapamycin-related chemical compound.

In still other embodiments, the present invention generates T cells (e.g., CTLs, helper T cells, or Tregs) expressing a chimeric antigen receptor. Cells can be efficiently transduced by a vector, such as but not limited to retroviral or nonintegrating viral vectors (e.g., adenoviral, adeno-associated viral, integration-deficient retro-lentiviral, poxviral) or nonviral vectors (e.g., plasmid vectors, artificial chromosomes) or episomal or episomal hybrid vectors) carrying a CAR targeting tumor antigen (e.g., CD19, CD38, CD33, CD47, CD20 etc.). CARs are designed to enhance a cells ability to recognize, bind to, and kill target cells (e.g., cancer or tumor cells). In some embodiments, the CAR enhances the cell's ability to recognize tumor cells. In some embodiments, the CAR enhances the cells anti-tumor activity. In some embodiments, and without limitation, the CAR is a G protein coupled receptor 87 (GPR87) CAR and solute carrier family 7 member 11 (SLC7A11 (xCT)) CAR, TNF receptor superfamily member 17 (BCMA) CAR, CD30 CAR, CD19 CAR, CD22-CAR, CD33 CAR, CD133-CAR, mesothelin-CAR, CD70 CAR, CD73 CAR, e.g. targeting the following tumors or tumor antigens:
(i) Human epidermal growth factor receptor 2 (HER2)-ovarian cancer, breast cancer, glioblastoma, colon cancer, osteosarcoma, and medulloblastoma;
(ii) Epidermal growth factor receptor (EGFR) positive malignancies, such as—non-small cell lung cancer, epithelial carcinoma, cholangiocarcinoma and glioma;
(iii) Mesothelin-mesothelioma, ovarian cancer, and pancreatic adenocarcinoma;
(iv) Prostate-specific membrane antigen (PSMA)—prostate cancer;
(v) Carcinoembryonic antigen (CEA)—pancreatic adenocarcinoma, breast cancer, and colorectal carcinoma;
(vi) Glypican-3—hepatocellular carcinoma;
(vii) Variant III of the epidermal growth factor receptor (EGFRvIII)—glioblastoma;
(viii) Disialoganglioside 2 (GD2)-neuroblastoma and melanoma;
(ix) Carbonic anhydrase IX (CAIX)—renal cell carcinoma;
(x) Interleukin-13Ra2—glioma;
(xi) Fibroblast activation protein (FAP)—malignant pleural mesothelioma;
(xii) L1 cell adhesion molecule (L1-CAM)—neuroblastoma, melanoma, and ovarian;
(xiii) Cancer antigen 125 (CA 125)—epithelial ovarian cancer;
(xiv) Cluster of differentiation 133 (CD 133)—glioblastoma and cholangiocarcinoma, adenocarcinoma;
(XV) Cancer/testis antigen 1B (CTAG1B)—melanoma and ovarian cancer;
(xvi) Mucin 1—seminal vesicle cancer;
(xvii) Folate receptor-a (FR-a)—ovarian cancer;
(xviii) Growth factor receptor selected from one or more of ErbB1, ErbB2, ErbB3, or ErbB4, IGF1R, IGF2R, TβR I-II, VEGFR1, VEGFR2, VEGFR3, PDGFR (α/β) or FGFR1 through 4.
(xix) EGFRvIII—Glioblastoma.
(xx) Claudin 18.2—solid tumors, advanced gastric adenocarcinoma, pancreatic adenocarcinoma.
(xxi) Mesothelin—mesothelioma, metastatic pancreatic, ovarian, cervical, lung.

See, for example, Zhou Z et al., Chimeric antigen receptor T cells applied to solid tumors. Front Immunol. 2022 Oct. 31; or Pooria et al, Novel antigens of CAR T cell therapy: New roads; old destination, Translational Oncology, Volume 14, Issue 7, 2021, Zhang C, et al., Chimeric Antigen Receptor T-Cell Therapy. In: StatPearls [Internet]. Treasure Island (FL): StatPearls Publishing, each of which is incorporated herein by reference.

In some embodiments, the CAR comprises an intracellular domain from the Fc epsilon receptor gamma (Fc epsilon RI gamma). However, in further contemplated embodiments the CAR may also comprise a T cell receptor (TCR) CD3 zeta (CD3zeta) intracellular domain, alone or in combination with additional components from second or third generation CAR constructs (e.g., CD28, CD134, CD137, and/or ICOS).

In some embodiments, the present invention generates T cells that exhibit T cell activation and subsequent T-cell mediated cytotoxicity to a similar degree as non-immunocompatibile T cells (e.g., Pan T cell lines, among other T cells used as experimental controls for evaluating T cell-mediated cytotoxicity). The T cells generated herein can exhibit a significant outperformance in T cell-mediated cytotoxicity in comparison to CD34+ derived T cells.

In another aspect, the disclosure provides a cell composition, which comprises a T cell population (or a precursor thereof, such as a progenitor T cell population) that is HLA-A^{neg}, HLA-DPB1^{neg}, and HLA-DQB1^{neg}. Despite such gene deletions and/or gene edits, the T cell composition retains full antigen presenting functionality and ability to differentiate from precursors (as described herein) as well as to more mature phenotypes. Cell compositions of this aspect provide advantages in HLA matching for a recipient, to avoid, for example, GVHD. In various embodiments, the T cell population is homozygous for both HLA-B and HLA-C. In some embodiments, the T cell population is homozygous for HLA-DRB1.

In some embodiments of this aspect, the T cell population is a T-progenitor cell population. In various embodiments, the T progenitor population engrafts in the thymus, spleen, or secondary lymphoid organ upon administration. In other embodiments, the T cell population is a cytotoxic T cell (CTL) population, a helper T cell population, or a Treg population. In various embodiments, the T cell population may express a chimeric antigen receptor (CAR). The cell populations according to this aspect may be prepared according to other aspects of this disclosure.

In aspects, the invention provides a cell population, or pharmaceutically acceptable composition thereof, as described herein or produced by the method described herein. In some embodiments, the cell population is a progenitor T cell population capable of engraftment in a thymus, spleen, or secondary lymphoid organ upon administration to a subject in need. In other embodiments, the cell population is an α/β T cell population, a CAR-T cell population, a CTL population (which can express a CAR), a T helper population, or a Treg population (each as described herein). In various embodiments, the composition for cellular therapy is prepared that comprises the cell population and a pharmaceutically acceptable vehicle. The pharmaceutical composition may comprise at least about 10² cells, or at least about 10³, or at least about 10⁴, or at least about 10⁵, or at least about 10⁶, or at least about 10⁷, or at least about 10⁸ cells, or at least about 10⁹ cells, or at least about 10¹⁰ cells, or at least about 10¹¹ cells, or at least about 10¹² cells, or at least about 10¹³ cells, or at least about 10¹⁴ cells. For example, in some embodiments, the pharmaceutical composition is administered, comprising T progenitors of from about 100,000 to about 400,000 cells per kilogram (e.g., about 200,000 cells/kg). In other embodiments, cells are administered at from about 10⁵ to about 5×10⁵ cells per kilogram (e.g., about 2.5×10⁵ cells/kg), or from about 10⁶ to about 5×10⁶ cells per kilogram (e.g., about 2.5×10⁶ cells/kg), or from about 5×10⁶ to about 10⁷ cells per kilogram (e.g., about 5×10⁶ cells/kg) or from about 10⁷ to about 10⁸ cells per kilogram (e.g., about 5×10⁷ cells/kg) or from about 10⁸ to about 10⁹ cells per kilogram (e.g., about 5×10⁸ cells/kg) or from about 10⁹ to about 10¹⁰ cells per kilogram or from about 10¹⁰ to about 10¹¹ cells or from about 10¹¹ to about 10¹² cells per kilogram or from about 10¹² to about 10¹³ cells per kilogram or from about 10¹³ to about 10¹⁴ cells per kilogram of a recipient's body weight.

The cell composition of this disclosure may further comprise a pharmaceutically acceptable carrier or vehicle suitable for intravenous infusion or other administration route, and the composition may include a suitable cryoprotectant. An exemplary carrier is DMSO (e.g., about 10% DMSO). Cell compositions may be provided in unit vials or bags and stored frozen until use. In certain embodiments, the volume of the composition is from about one fluid ounce to one pint.

In some embodiments, the cell population is a Treg population useful for adoptive cell therapy, for example, for human subjects having a condition selected from an autoimmune or inflammatory condition or disease or graft versus host disease (GVHD). Further, various genetic disorders can impact the immune system, manifesting as autoimmune or pro-inflammatory state. In some embodiments, the Treg population is a CAR-T cell. In various embodiments, the regulatory T cells may express a tissue or cell-specific CAR. That is, the cells may express a CAR specific for an organ or tissue of interest, such as pancreas, liver, skin, muscle, bone, joints, thyroid, nerves, etc. In some embodiments, the Tregs comprise a TCR or CAR targeting the cells to pancreatic islets, for example, for treatment of Type 1 diabetes or prediabetes.

In some embodiments, the cell populations are derived from autologous cells or universally compatible donor cells or HLA-modified or HLA null cells (e.g., as described herein). That is, the cell populations are generated from iPSCs that were prepared from cells of the recipient subject or prepared from donor cells (e.g., universal donor cells, HLA-matched cells, HLA-modified cells, or HLA-null cells).

In other aspects, the invention provides a method for cell therapy, comprising administering the cell population described herein, or pharmaceutically acceptable composition thereof, to a human subject in need thereof. In various embodiments, the methods described herein are used to treat blood (malignant and non-malignant), bone marrow, and immune diseases. In various embodiments, the human subject has a condition comprising one or more of lymphopenia, a cancer, infectious disease (e.g., viral disease such as HPV or HIV) an immune deficiency, an autoimmune disease, a skeletal dysplasia, hemoglobinopathies, an anemia, a bone marrow failure syndrome, and a genetic disorder (e.g., a genetic disorder impacting the immune system).

In some embodiments, the subject has cancer, such as a hematological malignancy or a solid tumor. In such embodiments, the subject is administered T progenitor cells or T cells having an anti-tumor specificity (such as CTLs recognizing tumor antigens).

In some embodiments, the subject has a condition selected from acute myeloid leukemia; acute lymphoblastic leukemia; chronic myeloid leukemia; chronic lymphocytic leukemia; myeloproliferative disorders; myelodysplastic syndromes; multiple myeloma; Non-Hodgkin lymphoma; Hodgkin disease; aplastic anemia; pure red-cell aplasia; paroxysmal nocturnal hemoglobinuria; Fanconi anemia; thalassemia major; sickle cell anemia; severe combined immunodeficiency (SCID); Wiskott-Aldrich syndrome; hemophagocytic lymphohistiocytosis; inborn errors of metabolism; severe congenital neutropenia; Shwachman-Diamond syndrome; Diamond-Blackfan anemia; and leukocyte adhesion deficiency. T cells generated using the methods described herein are administered to the subject e.g., by intravenous infusion. In some embodiments, the methods can be performed following myeloablative, non-myeloablative, or immunotoxin-based (e.g., anti-c-Kit, anti-CD45, etc.) conditioning regimes.

In other embodiments, the invention provides a method for cell therapy, comprising administering a Treg cell population described herein, or pharmaceutically acceptable composition thereof, to a human subject in need thereof. In various embodiments, the subject has an autoimmune, alloimmune, or inflammatory disease. In some embodiments, the subject is a tissue or organ transplant recipient, and in some embodiments the subject is a recipient of an allogeneic organ or tissue transplant. In some embodiments, the subject is experiencing or is at risk for GVHD. Organs that can be transplanted, for example, include the heart, kidneys, liver, lungs, pancreas, intestine, and/or thymus. Tissues for transplant can include, for example, bones, tendons (both referred to as musculoskeletal grafts), bone marrow or HSCs, cornea, skin, heart valves, nerves and/or veins. Kidneys, liver and the heart are the most commonly transplanted organs. Cornea and musculoskeletal grafts are the most commonly transplanted tissues.

In some embodiments, the subject has an autoimmune condition, which in some embodiments is selected from Type 1 diabetes, Rheumatoid arthritis (RA), Psoriasis or psoriatic arthritis, Multiple sclerosis, Systemic lupus erythematosus (SLE), Inflammatory bowel disease, Addison's disease, Graves' disease, Sjogren's Syndrome, Hashimoto's thyroiditis, Myasthenia gravis, autoimmune vasculitis, scleroderma, Hemolytic anemia, Pernicious anemia, and Goodpasture's syndrome.

In some embodiments, the subject has an immune condition such as celiac disease, Hyperimmunoglobulin E syndrome, and IPEX Syndrome.

As used herein, the term “about” means ±10% of the associated numerical value.

Certain aspects and embodiments of this disclosure are further described with reference to the following examples.

### EXAMPLES

### Example 1—ETV2 Over-Expression Increases the Yield of Hemogenic Endothelial Cells and Enhances the CD34+ Cell Formulation During iPSC Differentiation but does not Affect Pluripotency

### Methods

iPSCs were developed from hCD34+ cells by episomal reprogramming as known in the art and essentially as described in Yu, et al. Induced pluripotent stem cell lines derived from human somatic cells, Science 318, 1917-1920, (2007); and J. Yu, et al. Human induced pluripotent stem cells free of vector and transgene sequences. Science 324, 797-801, (2009). Embryoid Bodies and hemogenic endothelium differentiation was performed essentially as described in: R. Sugimura, et al., Haematopoietic stem and progenitor cells from human pluripotent stem cells. Nature 545, 432-438, (2017); C. M. Sturgeon, et al, Wnt signaling controls the specification of definitive and primitive hematopoiesis from human pluripotent stem cells. Nat Biotechnol 32, 554-561, (2014); J. Yu, et al. Induced pluripotent stem cell lines derived from human somatic cells. Science 318, 1917-1920, (2007); and J. Yu, et al. Human induced pluripotent stem cells free of vector and transgene sequences. Science 324, 797-801, (2009).

Briefly, hiPSC were dissociated and resuspended in media supplemented with L-glutamine, penicillin/streptomycin, ascorbic acid, human holo-Transferrin, monothioglycerol, BMP4, and Y-27632. Next, cells were seeded in 10 cm dishes (EZSPHERE or low attachment plate) for the EB formation. On Day 1, bFGF and BMP4 were added to the medium. On Day 2, the media was replaced with a media containing SB431542, CHIR99021, bFGF, and BMP4. On Day 4, the cell media was replaced with a media supplemented with VEGF and bFGF. On day 6, the cell media was replaced with a media supplemented with bFGF, VEGF, interleukin (IL)-6, IGF-1, IL-11, SCF, and EPO. Cells were maintained in a 5% CO₂, 5% O₂, and 95% humidity incubator. To harvest the CD34+ cells, the EBs were dissociated on day 8, cells were filtered through a 70 μm strainer, and CD34+ cells were isolated by CD34 magnetic bead staining.

### Results

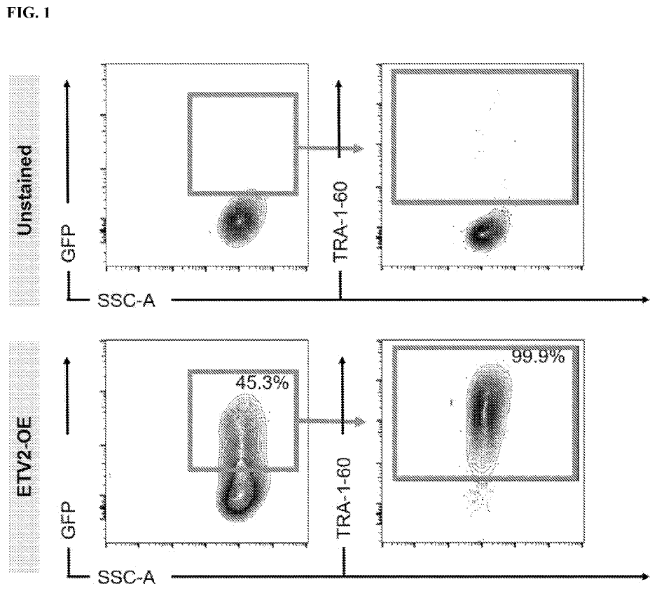

An adenoviral vector containing both ETV2 and GFP sequences under the control of the EF1A promoter was used to transduce induced pluripotent stem cells (iPSCs). After the transduction, about 45% of the iPSC culture was observed to be GFP positive, thus confirming ETV2 overexpression (ETV2-OE). It was further observed that ETV2-OE in iPSC cells preserves the pluripotency properties of iPSCs as shown by the stemness marker expression TRA-1-60 (FIG. 1). FIG. 1 shows FACS plots representative of transduction efficiency of iPSC with an adenoviral vector to overexpress the ETV2 and the GFP sequences.

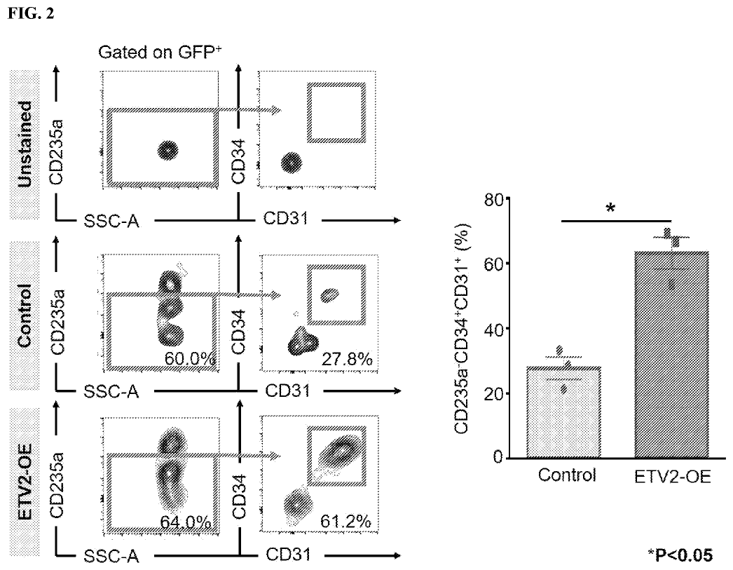

Next, the ETV2-OE-iPSCs were differentiated (along with control iPSCs transduced with a vector bearing the GFP sequence without ETV2) to embryoid bodies and subsequently to hemogenic endothelial cells (Strugeon et al., 2014). The results suggest that the overexpression of ETV2 boosts the formation of hemogenic endothelial cells as demonstrated by the expression of the CD34⁺ and CD31⁺ markers within the CD235a⁻ population (FIG. 2). Specifically, FIG. 2 shows representative flow cytometric analysis of hemogenic endothelial cells (defined here as CD235a−CD34+CD31+) and relative quantification demonstrates that ETV2-OE enhances the formation of hemogenic endothelial cells as compared to controls.

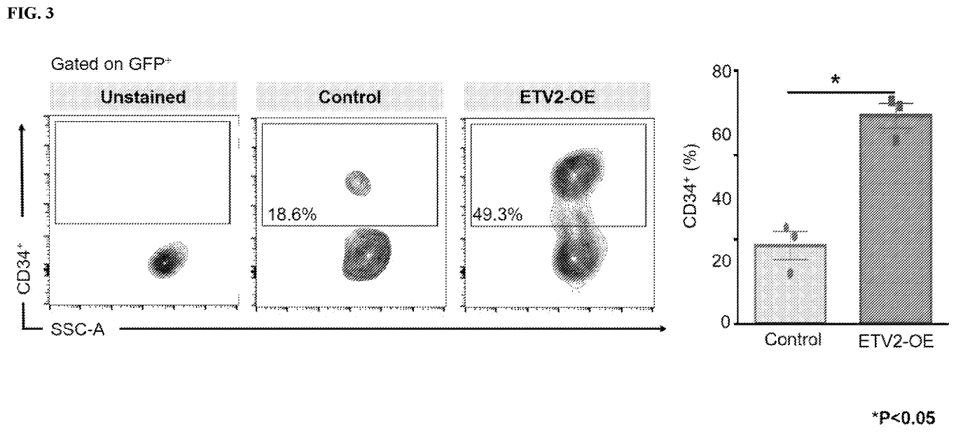

Moreover, the results suggest that ETV2-OE enhances the formation of the CD34⁺ cells (FIG. 3). FIG. 3 shows representative flow cytometric analysis of CD34+ cells and relative quantification demonstrates that ETV2-OE enhances the CD34+ cell formation.

Overall, these data indicate that ETV2 overexpression in iPSCs does not affect their pluripotency properties and facilitates their ability to undergo the hemogenic endothelial and hematopoietic differentiations.

### Example 2—iPSC-Derived HSCs Generated with Piezo1 Activation Undergo T Cell Differentiation Similar to Bone Marrow-Derived HSCs

### Methods

To analyze the EHT, EB-derived CD34+ cells were suspended in medium containing Y-27632, TPO, IL-3, SCF, IL-6, IL-11, IGF-1, VEGF, bFGF, BMP4, and FLT3. After the cells had adhered to the bottom of the wells for approximately 4-18 hours (by visual inspection), Yoda1 was added to the cultures for some experiments. After 4-7 days, the cells were collected for analysis.

iPSCs were differentiated to embryoid bodies for 8 days. At day 8, CD34+ cells from iPSC-derived embryoid bodies were harvested and cultured for additional 5 to 7 days to induce endothelial-to-hematopoietic (EHT) transition (with or without Yoda1). Then, CD34+ cells were harvested from the EHT culture between day 5 to day 7 for further hematopoietic lineage differentiation and analysis.

CD34+ cells, harvested from the EHT culture between day 5-7 (or total of day 13-21 differentiation from iPSCs), were seeded in 48-well plates pre-coated with rhDL4 and RetroNectin. T lineage differentiation was induced in media containing aMEM, FBS, ITS-G, 2BME, ascorbic acid-2-phosphate, Glutamax, rhSCF, rhTPO, rhIL7, FLT3L, rhSDF-1a, and SB203580.

Between day 2 to day 6, 80% of the media was changed every other day. At D7, cells were transferred into new coated plates and analyzed for the presence of pro-T cells (CD34+ CD7+ CD5+/−).

Between day 8 to day 13, 80% of the media was changed every other day. At D14, 100,000 cells/wells were transferred to a new coated plate and the cells analyzed for the presence of pre-T cells (CD34− CD7+ CD5+/−).

Between day 15 to day 20, 80% of the media was changed every other day. Cells were harvested at D21, and the cells were analyzed for CD3, CD4, CD8, CD5, CD7, TCRab expression, as surrogates for T cells, via FACS, and/or activated using CD3/CD28 beads to evaluate their functional properties.

After 21 days of differentiation, cells were collected and re-seeded at approximately 80,000 cells into new 96-well culture plates in RPMI 1640 (no L-glutamine; no phenol red) plus FBS, L-glutamine, IL-2, and then activated with 1:1 CD3/CD28 beads. After 72 hours of activation with CD3/CD28 beads, cells were analyzed for CD3, CD69, CD25 expression by FACS and IFN-γ expression using RT-qPCR. The supernatant was analyzed by ELISA.

### Results

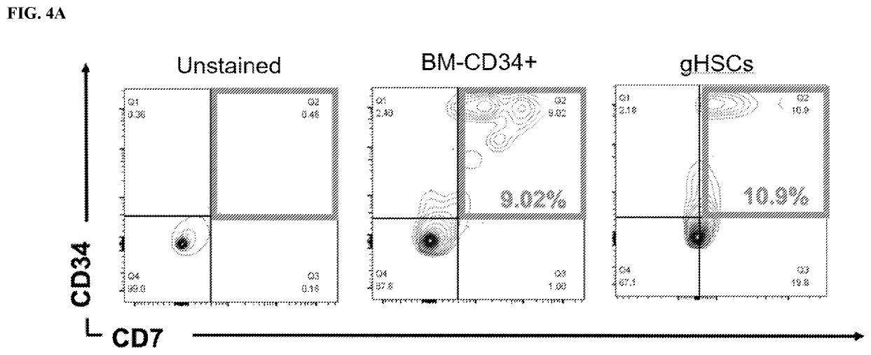

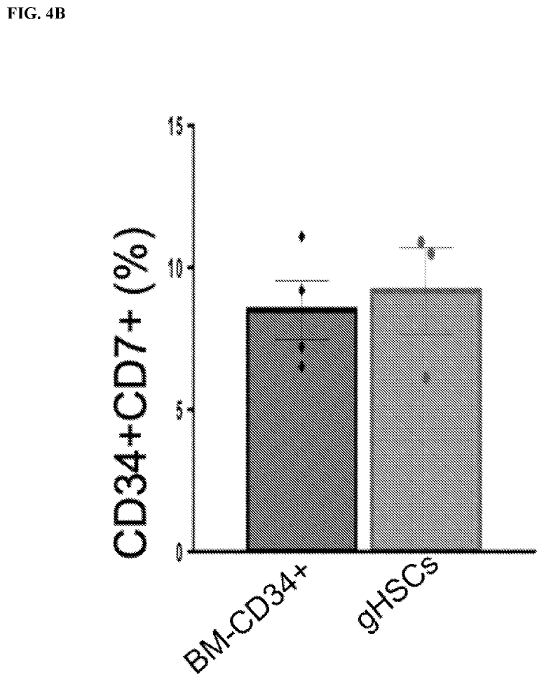

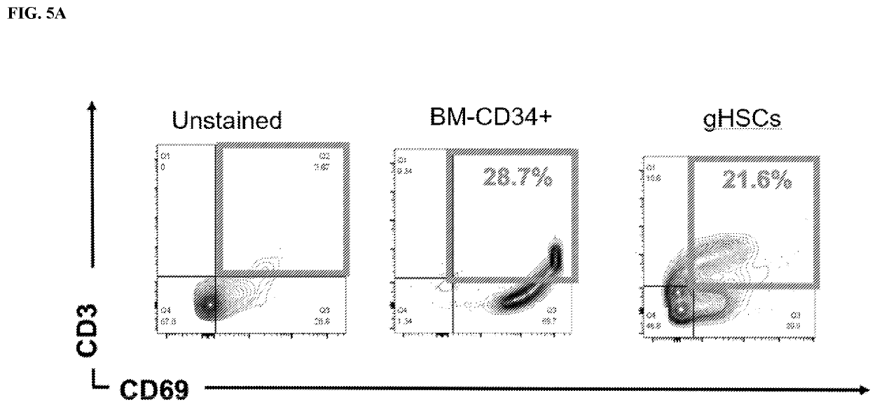

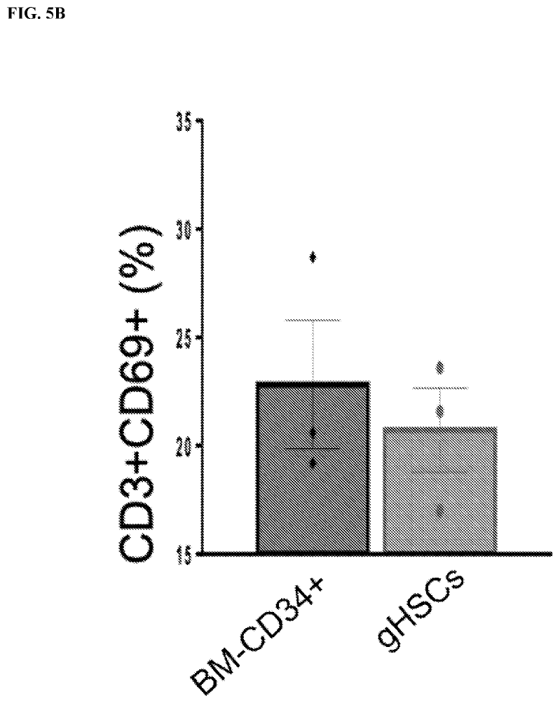

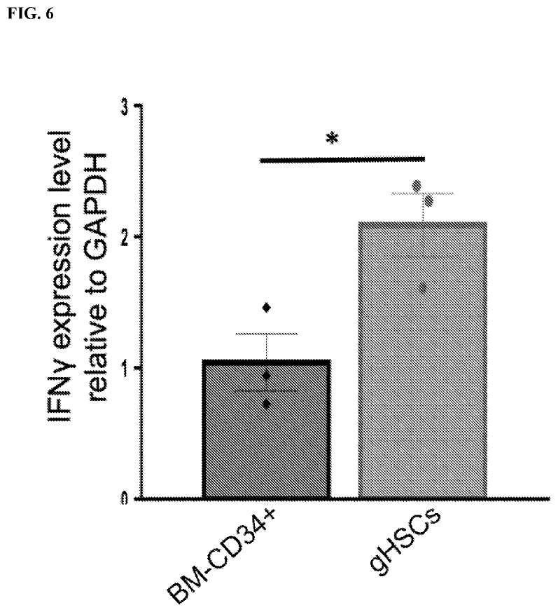

FIG. 4A and FIG. 4B show that iPSC-derived HSCs that are derived with EHT of CD34+ cells from differentiated iPSCs (e.g., in this case including Piezo1 activation) undergo pro-T cell differentiation similar to bone marrow (BM)-HSCs. Further, FIG. 5A and FIB. 5B show that iPSC-derived HSCs generated with EHT of CD34+ cells from differentiated iPSCs (in this case involving Piezo1 activation) undergo T cell differentiation and can be activated with CD3/CD28 beads similar to BM-HSCs. FIG. 6 shows that iPSC-derived HSCs (in this case generated with Piezo1 activation) can differentiate to functional T cells, as demonstrated by INFγ expression upon stimulation with CD3/CD28 beads. Together, these results demonstrate that iPSC-derived HSCs (i.e., derived with EHT of CD34+ cells from differentiated iPSCs) enhances HSC ability to further differentiate to progenitor T cells and functional T cells ex vivo. Experiments shown in FIG. 6 involve Piezo1 activation during HSC formation.

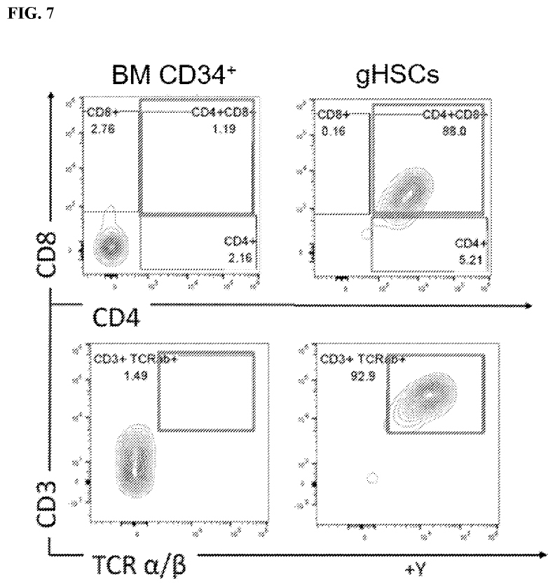

FIG. 7 shows that HSCs generated according to this disclosure (labeled as D8+7 iPSC-CD34+) successfully differentiate into CD4+CD8+ (“double positive”) T cells as well as TCR α/β T cells. The methods of the present disclosure substantially outperform bone marrow CD34+ cells for T cell maturation. FIG. 7 shows results with (“+Y”) and without (“-Y”) Yoda1 during HSC formation.

FIG. 8 shows that HSCs generated according to this disclosure (D8+7 iPSC-CD34+ cells (+ or − Yoda1) successfully rearrange TCR, and outperform bone marrow CD34+ cells. Shown are iPSC and EB negative controls, Peripheral Blood T cells as positive control, T cells generated from BM CD34+ cells, and T cells generated according to this instant disclosure with and without Yoda1.

### Example 3—Evaluating Off-Target Editing in HLA Knockout HSCs

HLA typing of the triple knockout (HLA edited) HSC clones was performed to check for unwanted editing and to ensure that no major editing events, e.g., deletion(s), occurred within other regions of chromosome 6. Sequencing methods and analyses were performed to evaluate the degree of gRNA off-target activity and to select gRNAs that represent a low risk of affecting non-target HLA genes.

Sequencing was performed by using in situ break labelling in fixed and permeabilized cells by ligating a full-length P5 sequencing adapter to end-prepared DSBs. Genomic DNA was extracted, fragmented, end-prepared, and ligated using a chemically modified half-functional P7 adapter. The resulting DNA libraries contained a mixture of functional DSB-labelled fragments (P5: P7) and non-functional genomic DNA fragments (P7: P7). Subsequent DNA sequencing of the DNA libraries enriched for DNA-labelled fragments, eliminating all extraneous, non-functional DNA. As the library preparation is PCR-free, each sequencing read obtained was equivalent to a single labelled DSB-end from a cell. This generated a DNA break readout, enabling the direct detection and quantification of genomic DSBs by sequencing without the need for error-correction and enabled mapping a clear list of off-target mutations.

Table 1 below summarizes the results of the editing strategy in two representative HLA edited clones relative to wild-type cells (gHSCs).

TABLE 1
Clonal HSC HLA knockouts.

| Sample ID | Locus | Allele 1 | Allele 2 | Comments |
|---|---|---|---|---|
| WT | A | A*01:01:01 | A*01:01:01 | Not affected |
| (gHSC) | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | DPB1*01:01:01 | DPB1*04:01:01 | Not affected |
| | DQA1 | DQA1*05:01:01 | DQA1*05:01:01 | Not affected |
| | DQB1 | DQB1*02:01:01 | DQB1*02:01:01 | Not affected |
| | DRB1 | DRB1*03:01:01 | DRB1*03:01:01 | Not affected |
| | DRB345 | DRB3*01:01:02 | DRB3*01:01:02 | Not affected |
| Clone #1 | A | xxx | xxx | Deletion in |
| | | | | Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPB1 | xxx | xxx | Deletion in |
| | | | | Exon 2 |
| | DQA1 | DQA1*05:01:01 | DQA1*05:01:01 | Not affected |
| | DQB1 | xxx | xxx | Delction in |
| | | | | Exon 2 |
| | DRB1 | DRB1*03:01:01 | DRB1*03:01:01 | Not affected |
| | DRB345 | DRB3*01:01:02 | DRB3*01:01:02 | Not affected |
| Clone #2 | A | xxx | xxx | Deletion in |
| | | | | Exon 2 |
| | B | B*08:01:01 | B*08:01:01 | Not affected |
| | C | C*07:01:01 | C*07:01:01 | Not affected |
| | DPA1 | DPA1*01:03:01 | DPA1*02:01:02 | Not affected |
| | DPBI | xxx | xxx | Deletion in |
| | | | | Exon 2 |
| | DQA1 | DQA1*05:01:01 | DQA1*05:01:01 | Not affected |
| | DQB1 | xxx | xxx | Deletion in |
| | | | | Exon 2 |
| | DRB1 | DRB1*03:01:01 | DRB1*03:01:01 | Not affected |
| | DRB345 | DRB3*01:01:02 | DRB3*01:01:02 | Not affected |

Table 2 provides a non-limiting example of gRNAs used in the experiments which can be used to knock out expression of indicated HLA genes.

TABLE 2
Exemplary gRNA sequences

| gRNA ID | Spacer sequence |
|---|---|
| HLA-A | CGTAGCCCACGGCGATGAAG (SEQ ID NO: 1) |
| | ATTTCTTCACATCCGTGTCC (SEQ ID NO: 2) |
| | GTCTCCTGGTCCCAATACTC (SEQ ID NO: 3) |
| | GTAGCCCACGGCGATGAAGC (SEQ ID NO: 7) |
| | TAGCCCACGGCGATGAAGCG (SEQ ID NO: 5) |
| | GAGGGTTCGGGGCGCCATGA (SEQ ID NO: 6) |
| | CCGGAACACACGGAATGTGA (SEQ ID NO: 7) |
| | ACAGCGACGCCGCGAGCCAG (SEQ ID NO: 8) |
| | CCACTCACAGATTGACCGAG (SEQ ID NO: 9) |
| | CGACGCCGCGAGCCAGAGGA (SEQ ID NO: 10) |
| | CAGATTGACCGAGTGGACCT (SEQ ID NO: 11) |
| | CAGACTGACCGAGTGGACCT (SEQ ID NO: 12) |
| | ACAGACTGACCGAGTGGACC (SEQ ID NO: 13) |
| | GCCGGGCCGGGACACGGATG (SEQ ID NO: 14) |
| | CCAGGAGACACGGAATGTGA (SEQ ID NO: 15) |
| | CCAGTCACAGACTGACCGAG (SEQ ID NO: 16) |
| | TCGACAGCGACGCCGCGAGC (SEQ ID NO: 17) |
| HLA-DPB1 | GAGATACATCTACAACCGGG (SEQ ID NO: 18) |
| | TGGAGAGATACATCTACAAC (SEQ ID NO: 19) |
| | GGTTGTAGATGTATCTCTCC (SEQ ID NO: 20) |
| | GGAGAGATACATCTACAACC (SEQ ID NO: 21) |
| | AGGAATGCTACGCGTTTAAT (SEQ ID NO: 22) |
| HLA-DQB1 | CAGATACATCTATAACCGAG (SEQ ID NO: 23) |
| | CGAGTACTGGAACAGCCAGA (SEQ ID NO: 24) |
| | CCCGTTGGTGAAGTAGCACA (SEQ ID NO: 25) |
| | GTGCTACTTCACCAACGGGA (SEQ ID NO: 26) |
| | AGGTCGTGCGGAGCTCCAAC (SEQ ID NO: 27) |
| | AGGTGACAGTTGCTACCCGA (SEQ ID NO: 28) |
| | TCCCGTTGGTGAAGTAGCAC (SEQ ID NO: 29) |
| | AACTACGAGGTGGCGTACCG (SEQ ID NO: 30) |
| | CTGTTCCAGTACTCGGCAAC (SEQ ID NO: 31) |
| | ACTACGAGGTGGCGTACCGC (SEQ ID NO: 32) |
| | CGAAGCGCACGTACTCCTCT (SEQ ID NO: 33) |

The results show that the editing strategy was successful in selectively targeting the HLA-A, DPB1, and DQB1 genes without affecting the other HLA genes or introducing major deletion elsewhere.

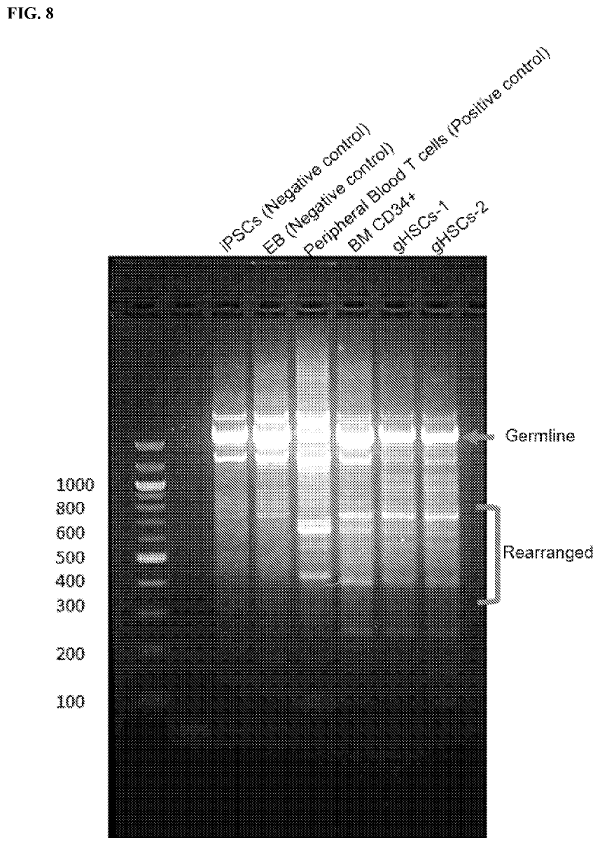

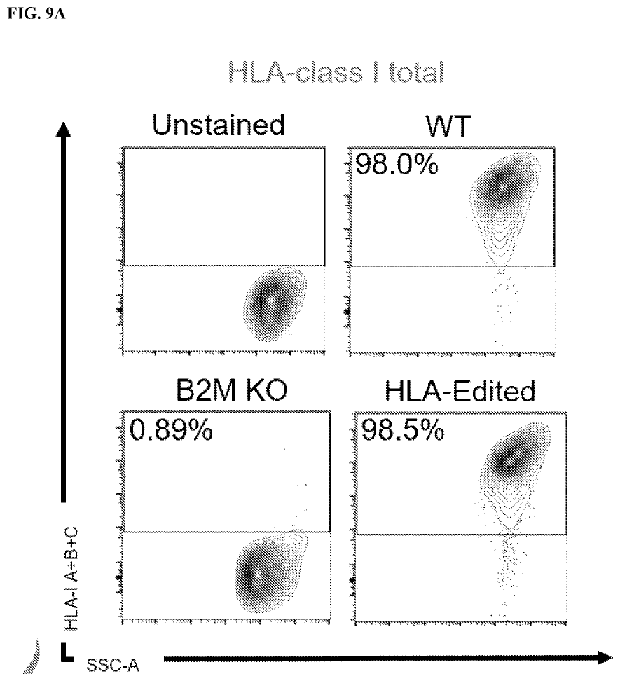

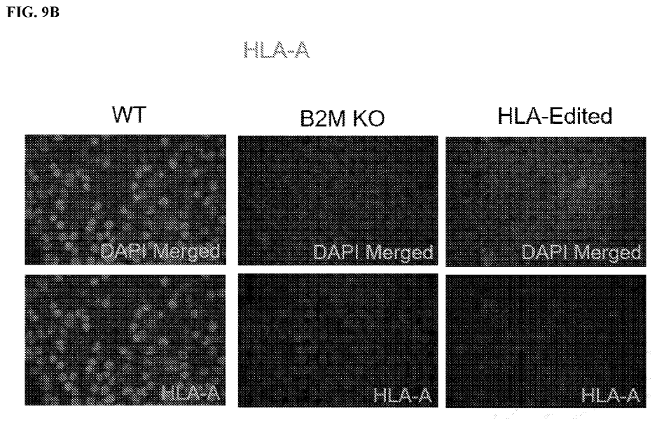

These results were confirmed by a phenotypic analysis of the HLA edited clones by FACS and immunofluorescence. As shown in FIGS. 9A and B, HLA edited cells tested positive for overall expression of HLA class-I molecules, comparable to the overall expression of HLA class-I molecules of wild-type cells (gHSCs). Specific expression of HLA-A via immunofluorescence confirmed that HLA-A was not expressed in the HLA edited cells, corroborating the finding that the gene editing strategy was successful in deleting only the HLA-A gene. Specifically, FIG. 9A, shows that the HLA edited cells were all positive for class-I like HLA to the same extent as the wild type cells (i.e., gHSC). This result indicates that despite the deletion of HLA-A, other class-I molecules like HLA-B and C were expressed and not affected by the gene editing strategy.

To confirm that the HLA-A gene was deleted, specific expression of the HLA-A was analyzed with immunofluorescence. As can be seen in FIG. 9B, HLA-A was not expressed in the HLA edited clone indicating that the gene editing strategy was efficient in specifically deleting the HLA-A gene only. Such preservation of overall class-I expression with deletion of HLA-A will facilitate patient matching while avoiding NK-cell mediated rejection.

### Example 4—Evaluating Pluripotency and Immunocompatibility of HLA Edited HSCs

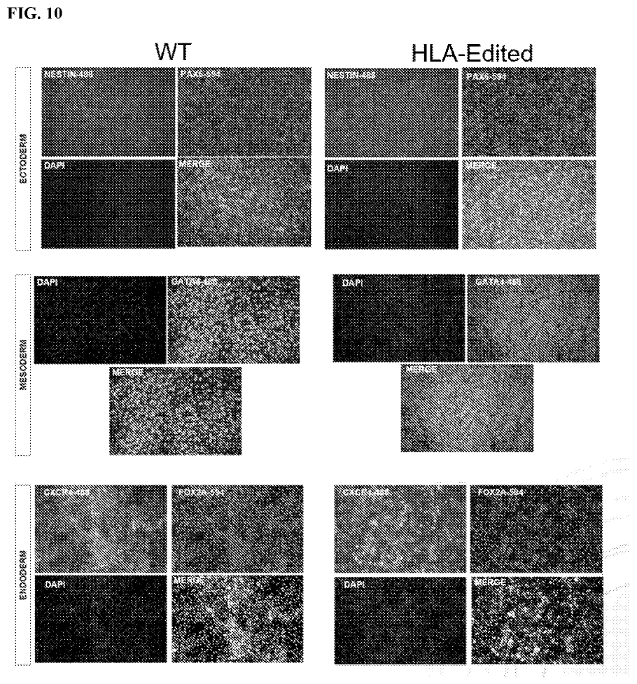

The ability of HLA edited cells to preserve pluripotency was evaluated. As shown in FIG. 10, immunofluorescence evaluation of the HLA edited iPSC clones indicated that they maintained trilineage differentiation, with ectoderm differentiation indicated by NESTIN-488 and PAX6-594 staining, mesoderm differentiation indicated by GATA-488 staining, and endoderm differentiation indicated by CXCR4-488 and FOX2A-594 staining.

HLA class I molecules are expressed on the surface of all nucleated cells and if the HLA class I molecules are mismatched between donor and recipient, then the cells could be recognized and killed by CD8+ T cells. Additionally, HLA mismatching could lead to cytokine release syndrome (CRS) and graft-versus-host disease (GVHD). Conversely, the complete deletion of HLA-I molecules, via B2M KO, would make the cell a target of NK cell-mediated cytotoxicity. The preservation of overall class-I expression with deletion of HLA-A can facilitate patient matching while preventing the NK-cell mediated rejection. Thus, the immunocompatibility of the HLA edited HSCs was tested by co-culture with peripheral blood mononuclear cells (PBMCs) to evaluate if the immune cells would reject a graft of the HLA edited and wild type HSCs (gHSCs).

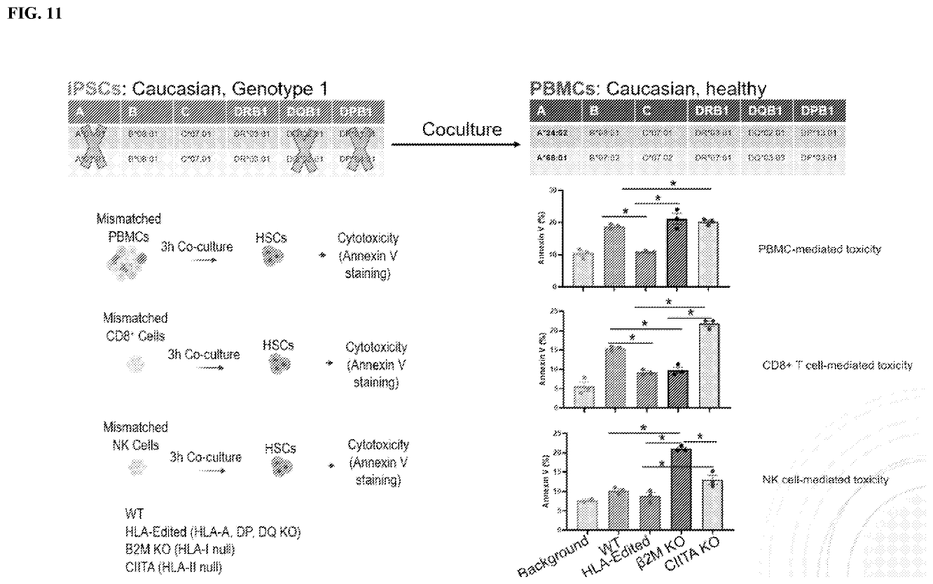

Wild-type (gHSCs) and HLA edited HSCs were co-cultured with PBMCs matching the HLA-B and HLA-C markers, but with mismatched HLA-A. B2M KO HSCs lacking expression of HLA class-I molecules and CIITA KO HSCs lacking expression of class-II molecules were used as controls to compare the degree of PBMC-mediated cytotoxicity for HLA-null and mismatched HLA, respectively. FIG. 11 shows the results of the PBMC-mediated cytotoxicity assay in the co-cultures as measured by an annexin V staining. The results show that deletion of HLA-A in the HLA edited HSCs protects the cells from PBMC-mediated cytotoxicity, while WT, B2M KO, and CIITA KO were susceptible to PBMC-mediated cytotoxicity. Co-cultured HSCs with sorted CD8+ T cells from the same PBMC donor protected HLA edited and B2M KO HSCs from CD8+ T cell cytotoxicity. Conversely, co-cultured HSCs with sorted NK cells only protected the WT and HLA edited cells from the NK cell-mediated cytotoxicity.

In summary, the immune compatibility results show that the CD8+ T cells present in the PBMC samples were responsible for killing the cells with mismatched HLA molecules (WT) and CIITA KO, while the NK cells present in the PBMCs were responsible for killing the HLA-null cells (B2M KO). However, HLA edited HSCs are protected from CD8+ T cell-mediated cytotoxicity (because the mismatched HLA-A had been knocked out), and protected from NK cell-mediated cytotoxicity (because HLA class I molecule expression was largely preserved).

### Example 5—Evaluating the In Vivo Engraftment Potential of HLA Edited HSCs

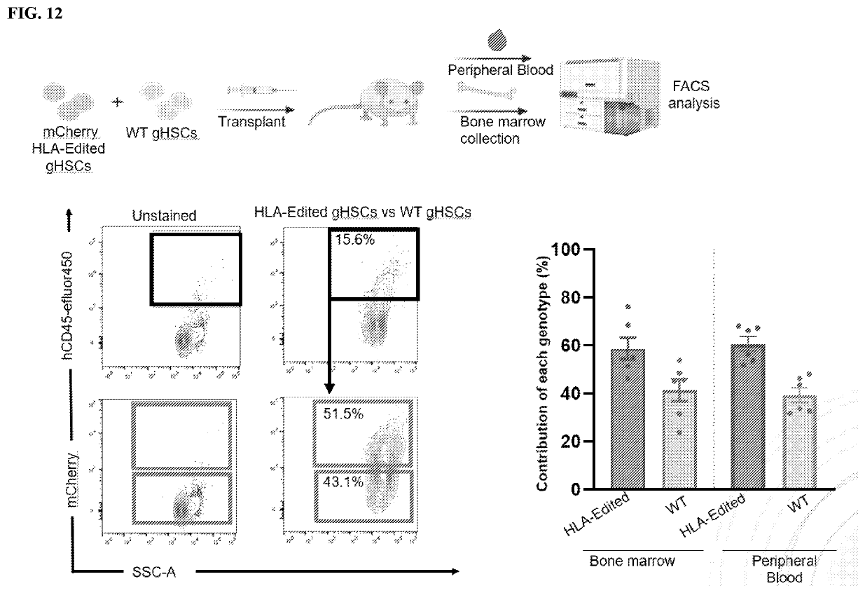

To evaluate the engrafting potential of HLA edited HSCs, the cells' ability to engraft in vivo was evaluated by a competitive transplant against WT HSCs. Equal proportions of mCherry HLA edited HSCs and wild-type HSCs (gHSCs) were admixed and transplanted into mice, from where bone marrow (BM) and peripheral blood samples were recovered and evaluated by FACS to compare the relative amounts of each cell type present in the samples. As shown in FIG. 12, both the HLA edited HSCs and the WT HSCs contributed to approximately equal engraftment in the BM and peripheral blood samples. These results confirm that HLA edited HSCs (prepared according to this disclosure) are comparable to WT HSCs in their engraftment and reconstitution potential. Hence, it is expected that properties of the WT HSCs are consistent with that of the HLA edited HSCs of the present disclosure, for generating T cell lineages.

### Example 6—Differentiation of HLA Edited HSCs to CD4+/CD8+ T Cells

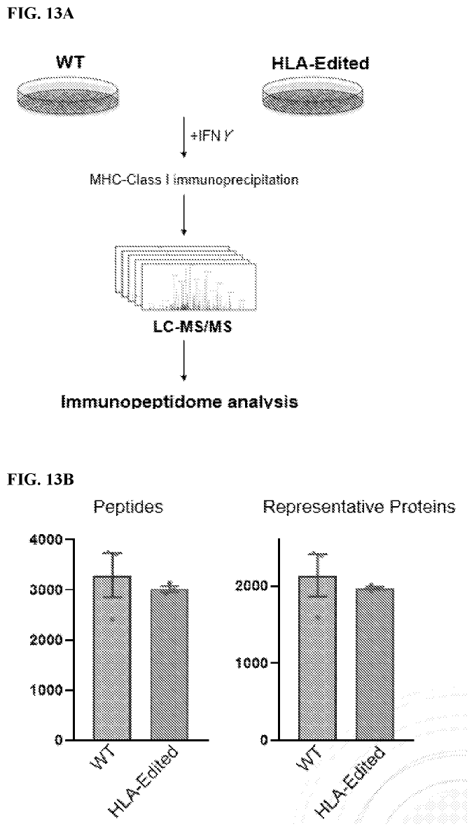

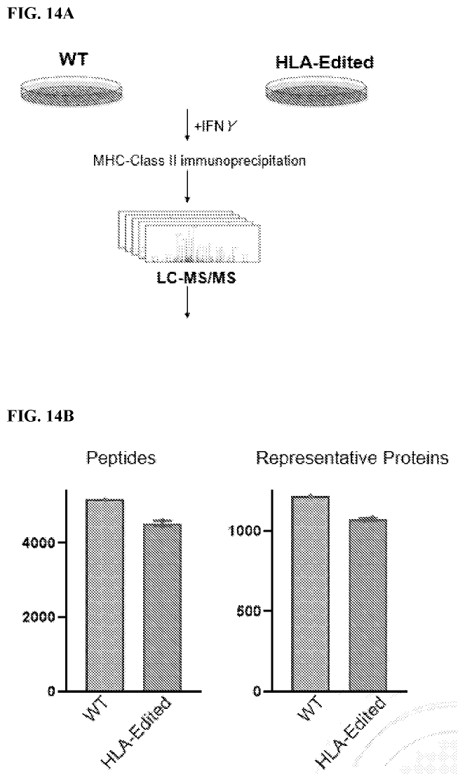

Antigen presenting cells (APCs) present antigens to helper CD4+ T cells through the HLA-II molecules. Activation of helper CD4+ T cells promotes the generation of antigen-specific CD8+ T cells which further develop into antigen-specific CTLs. Likewise, HLA Class I molecules are expressed on the surface of all nucleated cells and display peptide fragments of proteins from within the cell to CD8+ CTLs. CTLs induce cytotoxic killing of target (infected) cells upon recognition of HLA-I-peptide complex expressed on the cell surface. Hence, a study was carried out to determine if deletion of HLA-A impacts the edited HSCs' class I peptide presentation. As shown in FIGS. 13A and B, immunopeptidome analysis shows that the deletion of HLA-A does not impact overall class I peptide presentation. HLA-A edited cells showed comparable peptide and protein presentation when compared to wild type HSCs (gHSCs). Further, as shown in FIGS. 14A and B, deletion of HLA-DQB1 and HLA-DPB1 does not impact overall class II peptide presentation by macrophages differentiated from the HSCs. Together, these data suggest that despite the deletion of HLA-A, HLA-DQ, and HLA-DP molecules, the cells (and their derived lineages) preserve their ability to present a broad spectrum of class I and II peptides.

### Example 7—In Vivo Testing of Antigen-Mediated Immune Response

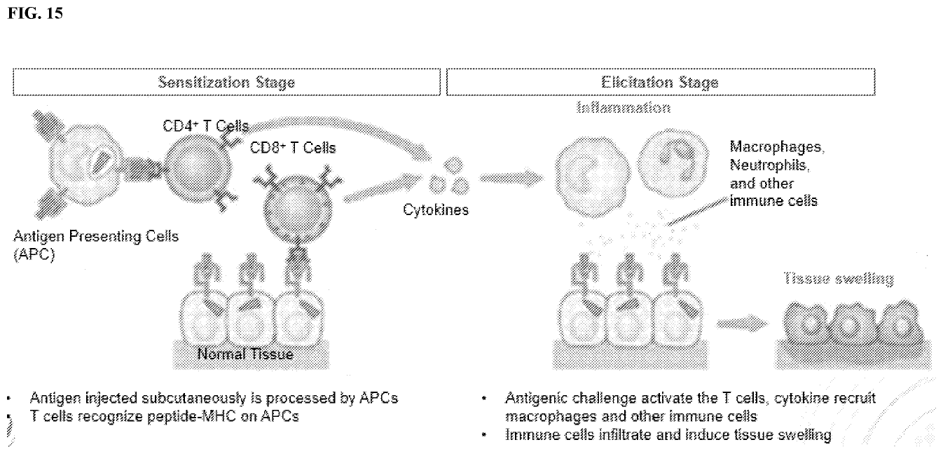

FIG. 15 is a schematic illustration of a Delayed Type Hypersensitivity Reaction, showing the sensitizing and eliciting stages of an antigen presentation. Briefly, upon antigen injection, antigen is processed by antigen presenting cells (APC) and presented by MHC Class II molecules on the APC surface. CD4+ T cells recognize peptide-MHC on antigen presenting cells (APCs). Upon antigenic challenge CD4+ helper T cells are activated and cytokines recruit macrophages and other immune cells, which induce tissue swelling.

A delayed-type hypersensitivity assay was performed on transplanted mice. Specifically, the mice were sensitized by subcutaneous injection of sheep Red blood cells as antigen. If the mice have a functional immune system, the APCs process the antigen and present peptide antigens to CD4+ T cells. Next, the mice were challenged by subcutaneous injection of the same antigen in the left paw. At this point the T cells are activated and secrete cytokines which recruit macrophages and other immune cells at the site of antigen injection creating tissue swelling. In this assay, a functional immune system resulted in the swelling of the left paw as measured with a micro caliper.

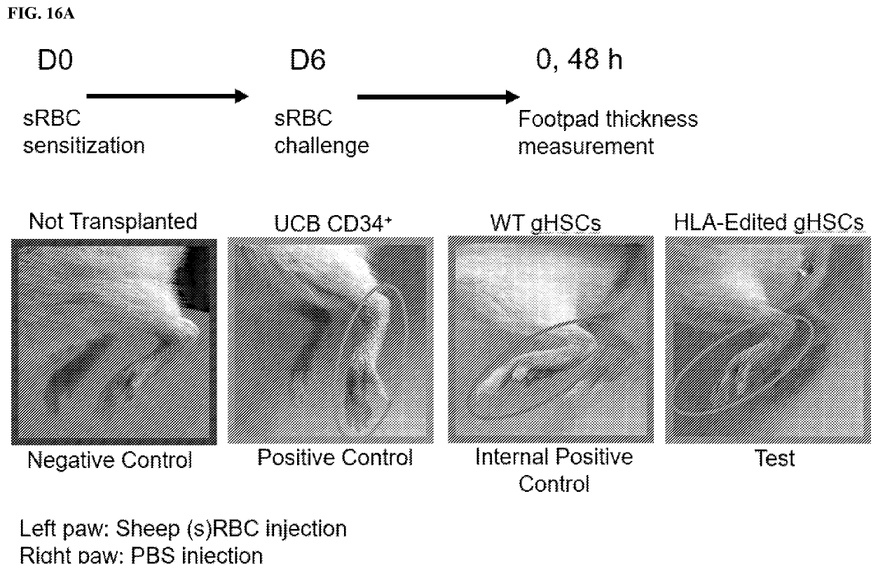

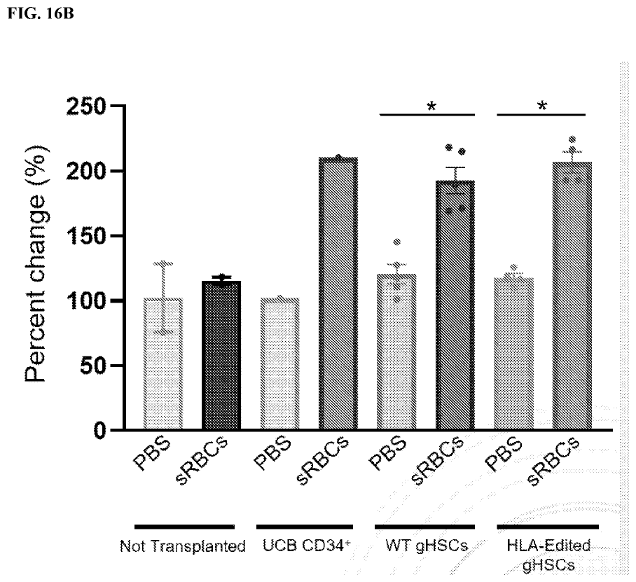

As can be seen in FIGS. 16A and B, the control (non-transplanted) mice did not show any left paw swelling as they are immunodeficient. Conversely, the mice transplanted with Cord Blood CD34⁺ cells showed tissue swelling and doubled the diameter of their left paw. A similar immune system response was found in both the mice transplanted with the WT (non-edited HSCs) and the HLA-edited HSCs (HLA edited).

### Example 8—Evaluating HSC-Derived Assorted T Cells and Pro-T Cell

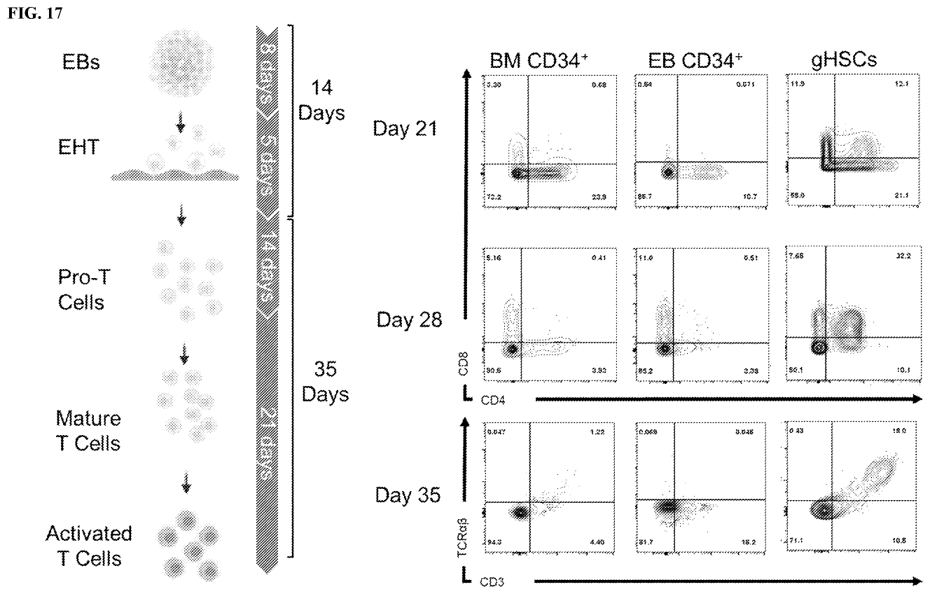

Next, the ability of the HSC-derived assorted T cells and pro-T cells to differentiate into mature T cells was tested. After a 35-day differentiation period, T cell precursors and derivatives thereof, were evaluated by cell sorting for the presence of CD4+, CD8+, and AB+ T cell populations. As shown in FIG. 17, pro-T cells differentiated into CD4+, CD8+, and αβ+ T cells more efficiently than bone marrow (BM)-derived CD34+ cells and the CD34+ cells derived from the embryonic bodies (EB).

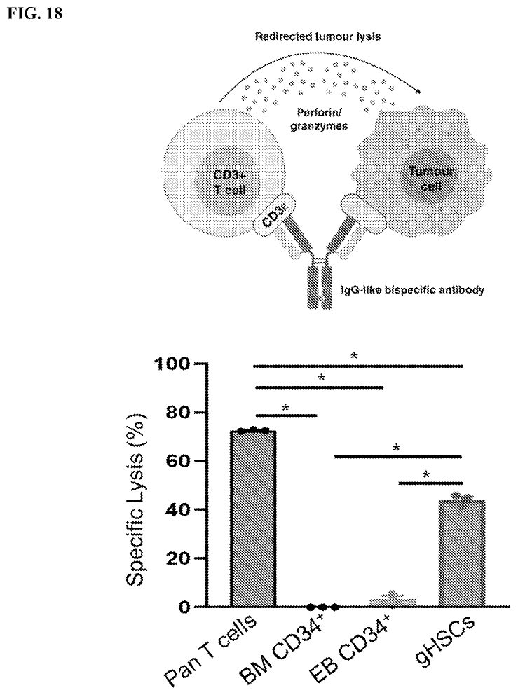

Next, to test the functional properties, each of the T cell populations were co-cultured with a CD19+ lymphoma cell line and an anti-CD3/CD-19 bispecific antibody. In this experimental model, the bispecific antibody engaged both the CD3 receptor on T cells and the CD19 cell surface receptor of the lymphoma cells, thus triggering T cell activation. The degree of activation was evaluated by measuring the subsequent T-cell mediated cytotoxicity in comparison to a Pan T cell positive control. As shown in FIG. 18, the T cells exhibited a statistically significant outperformance in cytotoxicity in comparison to both the BM CD34+ T cells and the EB CD34+ T cells.

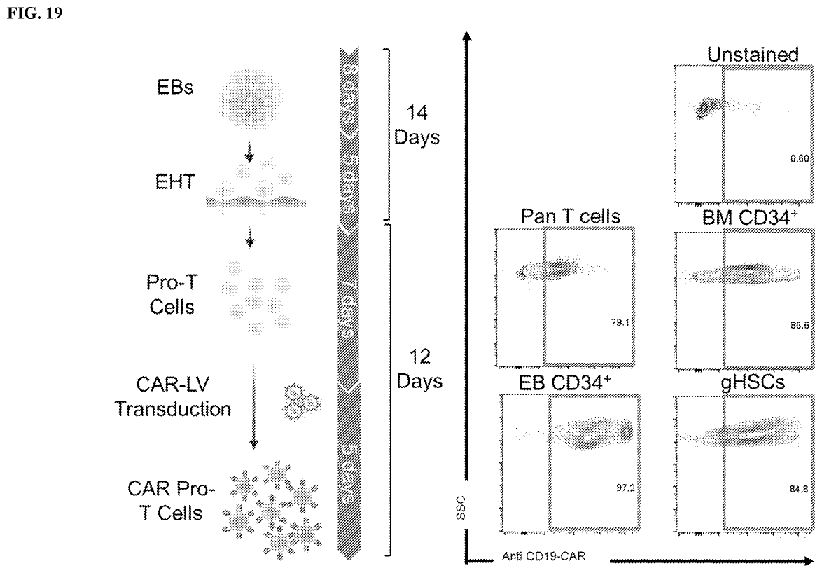

Because the overall differentiation process of T cells is 35 days long, a transduction experiment was performed to test if the time required to differentiate the HSCs could be decreased. Pro-T cells were cultured in an activation media (for approx. 7 days) to increase the transduction efficiency of the cells. Next, the cells were transduced with lentiviral (LV) particles encoding an anti-CD19 CAR transgene. The cells were cultured for additional 4-5 days (a total of 12 days) and their maturation and killing capabilities were evaluated. As shown in FIG. 19, the HSC-derived pro-T cells can be transduced with high efficiency, with more than 80% of the cells express the anti-CD19 CAR as evidenced by cell sorting.

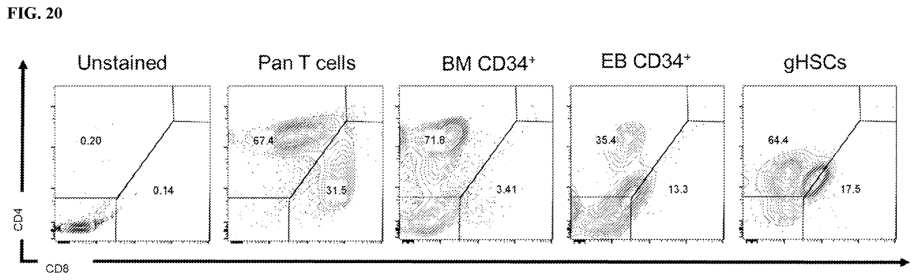

Next, the pro-T cells were evaluated for their ability to effectively mature into CD4+/CD8+ T cells via CAR transduction. The pro-T cells, along with bone marrow (BM)-derived CD34+ cells and CD34+ cells derived from the embryonic bodies (EB) (and Pan T cells as a positive control), underwent LV-transduction with the anti-CD19 CAR. The T cell subsets were screened by cell sorting for the presence of CD4 or CD8 cell surface marker expression. As shown in FIG. 20, the results indicated that CAR transduction promoted T cell maturation and that an increased degree of T cell maturation was observed in the pro-T cells in comparison to bone marrow (BM)-derived CD34+ cells and the CD34+ cells derived from the embryonic bodies (EB).

The ability of LV-transduced pro-T cells to function via anti-CD19 receptor-mediated cytotoxicity was evaluated. The T cell subsets were cocultured with a CD19+ leukemia cell line (NALM6) expressing a luciferase reporter gene (Luc+) to measure the degree of T cell-mediated cell lysis, with untransduced cells and Pan T cells as a negative and positive control, respectively. As shown in FIG. 21, the CAR T cells effectively functioned via T cell-mediated lysis, demonstrating a degree of cytotoxicity comparable to the CAR-pro T cells derived from the BM CD34+ cells. Conversely, the CAR T cells derived from the EB CD34+ cells showed no ability to kill the target cells.

### Example 9—Evaluating HSC Properties of Developing into Pro-T Cells

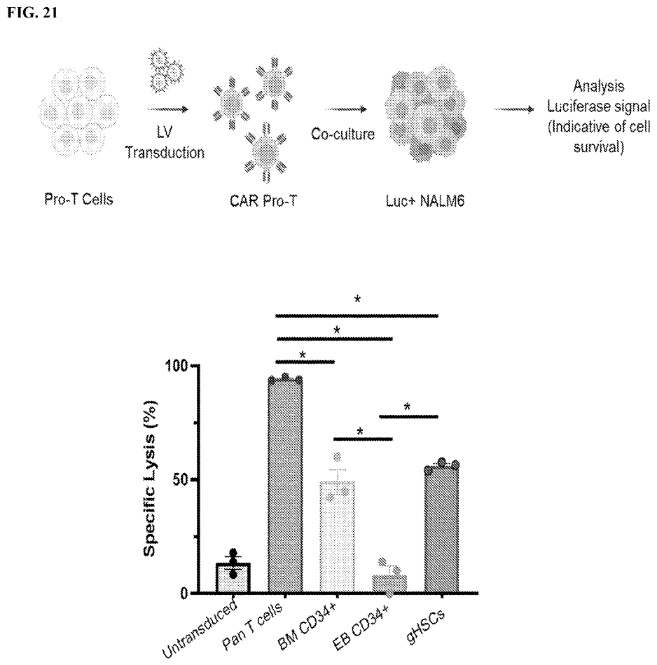

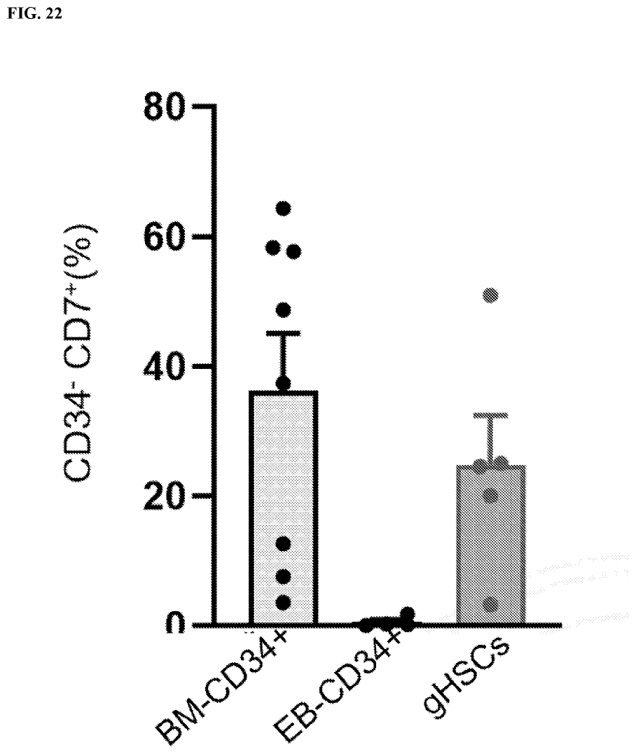

The ability of the HSCs to develop into pro-T cells was assessed by measuring the CD34−CD7+ markers on the pro-T cells. As shown in FIG. 22, FACS analysis showed that HSCs produced according to this disclosure successfully differentiated into CD34−CD7+ pro-T cells, as compared to bone marrow derived CD34+ cells or EB-derived CD34+ cells.

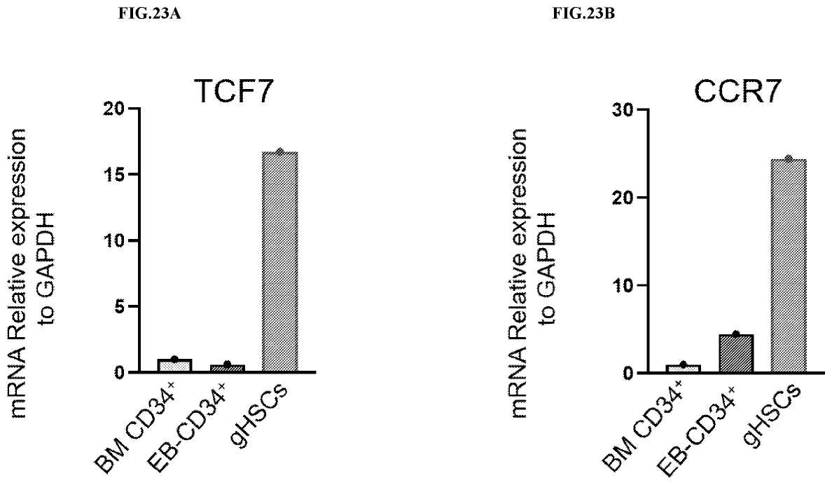

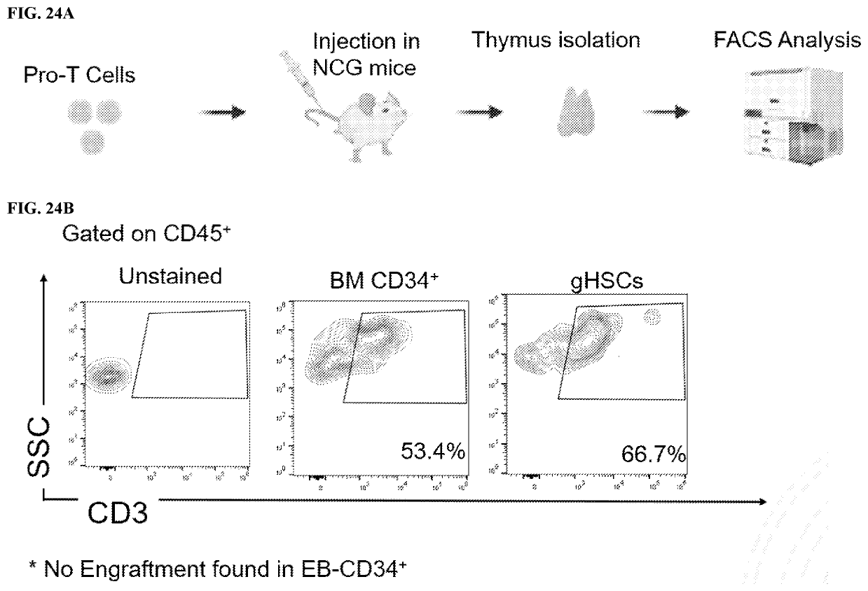

Next, the expression of T cell-specific transcription factors and thymus engrafting molecules were measured. FIG. 23A shows increased TCF7 expression and FIG. 23B shows increased CCR7 expression in the HSC-derived pro-T cells of the disclosure. FIG. 24A shows HSCs-derived Pro-T Cells engraft and differentiate in thymus. FIG. 24B shows FACS analysis of CD3 cell population of cells gated on a CD45+ cell population, which shows the superior engraftment and differentiation potential of the HSCs-derived Pro-T Cells in the thymus. Pro-T Cells of this example were prepared from HSCs using Piezo1 activation as already described.

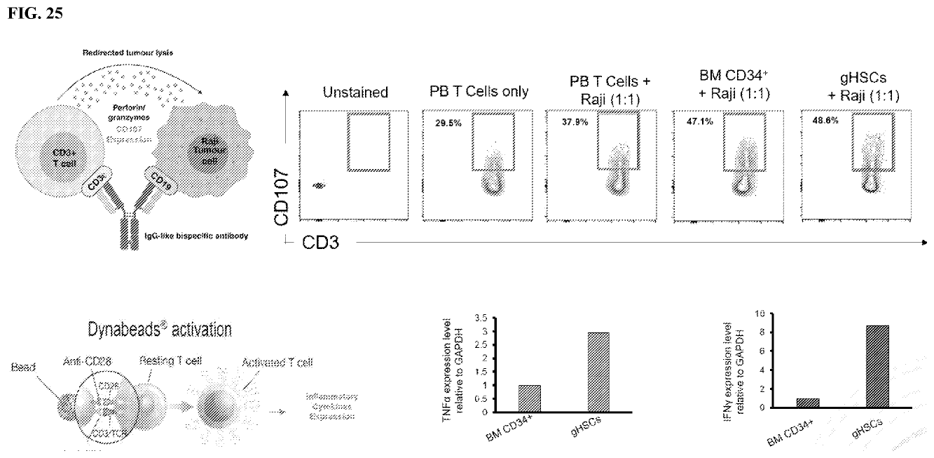

An in-vitro activation of the HSC-derived T cells were also measured, as illustrated in FIG. 25. Top panel of FIG. 25 shows FACS analysis of activated T cells from different sources, including the HSCs of the present disclosure (e.g., prepared using Piezo1 activation). T cells prepared from HSCs of the present disclosure demonstrated comparable or superior activation as measured by increased CD107 expression. The lower panel shows Dynabeads activation, where activated T cells express inflammatory cytokines. HSC-derived T cells according to the present disclosure (e.g., prepared using Piezo1 activation) expressed higher levels of inflammatory cytokines as exemplified by TNF-alpha and interferon gamma expression levels.

### Example 10—Evaluating Properties of CCR5 Knock Out HSCs to Develop into Pro-T Cells

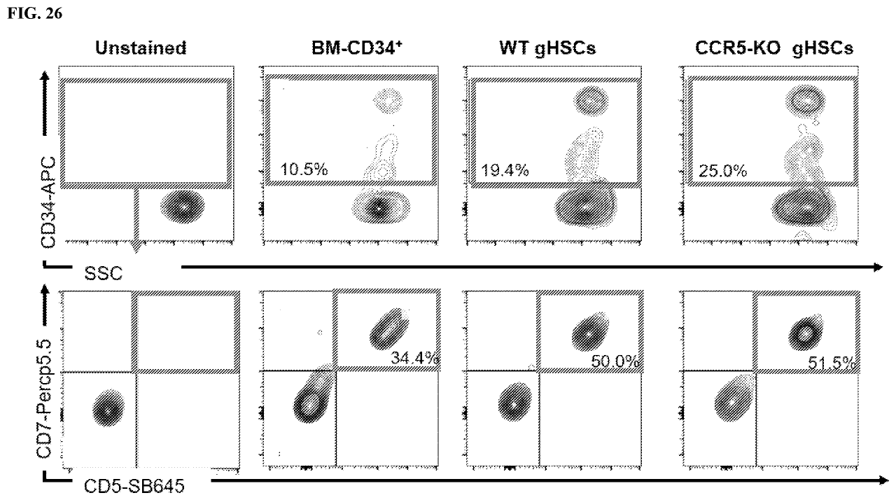

To determine if CCR5 knock out (CCR5-KO) HSCs can comparably differentiate to pro-T cells as their wild-type counterparts from which they are derived (i.e., HSCs of the present disclosure), a study was performed in which the CD34, CD7 and CD5 expression of the HSCs and the CCR5-KO were measured. As can be seen in FIG. 26, HSCs successfully differentiated into CD34+CD7+ CD5+ pro-T cells comparably to bone marrow derived CD34+ cells. Likewise, the CCR5-KO, like their wild type counterpart, successfully differentiated into CD34+CD7+CD5+ pro-T cells.

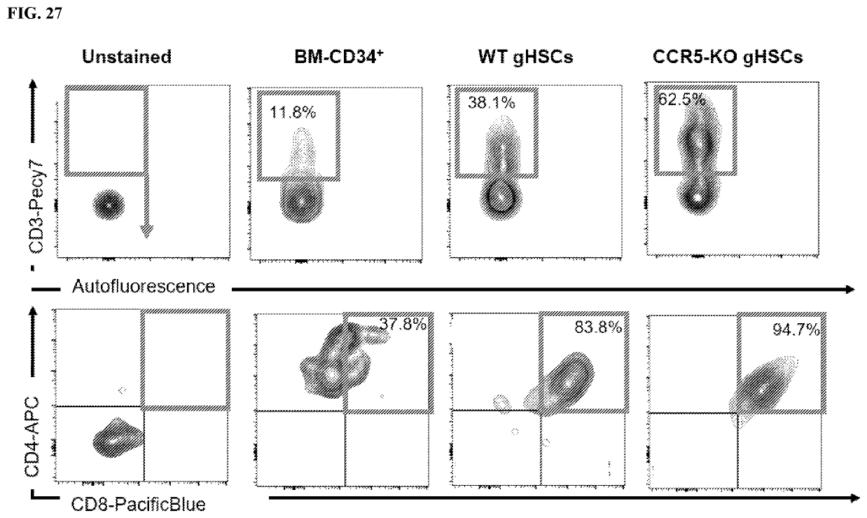

Next, the property of CCR5-knocked out HSCs to differentiate into double positive (CD4+CD8+) T cells was assessed and was it comparable to the HSCs from which they were derived. As can be seen in FIG. 27, CCR5-knocked out HSCs comparably differentiated into double positive (CD4+CD8+) T cells when compared to their wild type counterparts from which they were derived (i.e., HSCs of the present disclosure).

### REFERENCES

1. Nianias, A. & Themeli, M. Induced Pluripotent Stem Cell (iPSC)-Derived Lymphocytes for Adoptive Cell Immunotherapy: Recent Advances and Challenges. Curr Hematol Malig Rep 14, 261-268 (2019).
2. Brauer, P. M., Singh, J., Xhiku, S. & Zúñiga-Pflücker, J. C. T Cell Genesis: In Vitro Veritas Est? Trends Immunol 37, 889-901 (2016).
3. Kennedy, M. et al. T Lymphocyte Potential Marks the Emergence of Definitive Hematopoietic Progenitors in Human Pluripotent Stem Cell Differentiation Cultures. Cell Reports 2, 1722-1735 (2012).
4. Sturgeon, C. M., Ditadi, A., Awong, G., Kennedy, M. & Keller, G. Wnt Signaling Controls the Specification of Definitive and Primitive Hematopoiesis From Human Pluripotent Stem Cells. Nat Biotechnol 32, 554-561 (2014).
5. Chang, C.-W., Lai, Y.-S., Lamb, L. S. & Townes, T. M. Broad T-Cell Receptor Repertoire in T-Lymphocytes Derived from Human Induced Pluripotent Stem Cells. PLOS One 9, (2014).
6. Nishimura, T. et al. Generation of Rejuvenated Antigen-Specific T Cells by Reprogramming to Pluripotency and Redifferentiation. Cell Stem Cell 12, 114-126 (2013).
7. Themeli, M. et al. Generation of tumor-targeted human T lymphocytes from induced pluripotent stem cells for cancer therapy. Nat Biotechnol 31, 928-933 (2013).
8. Vizcardo, R. et al. Regeneration of Human Tumor Antigen-Specific T Cells from iPSCs Derived from Mature CD8+ T Cells. Cell Stem Cell 12, 31-36 (2013).
9. Montel-Hagen, A. et al. Organoid-induced differentiation of conventional T cells from human pluripotent stem cells. Cell Stem Cell 24, 376-389.e8 (2019).
10. Guo, R. et al. Guiding T lymphopoiesis from pluripotent stem cells by defined transcription factors. Cell Research 30, 21-33 (2020).
11. Nagano, S. et al. High Frequency Production of T Cell-Derived iPSC Clones Capable of Generating Potent Cytotoxic T Cells. Molecular Therapy—Methods & Clinical Development 16, 126-135 (2020).
12. Iriguchi, S. et al. A clinically applicable and scalable method to regenerate T-cells from iPSCs for off-the-shelf T-cell immunotherapy. Nature Communications 12, 430 (2021).

1. A method for preparing a T cell population or progenitors thereof, the method comprising:
enriching for CD34+ cells from a differentiated pluripotent stem cell (PSC) population to prepare a CD34+-enriched population;
inducing endothelial-to-hematopoietic transition of the CD34+-enriched cell population for at least two days, but no more than 12 days, to prepare a population comprising hematopoietic stems cells (HSCs) and/or hematopoietic stem progenitor cells (HSPCs); and
differentiating the population comprising HSCs and/or HSPCs to a progenitor T cell population or a T cell population.

2. (canceled)

3. The method of claim 1, wherein the population comprising HSCs and/or HSPCs is differentiated to a population comprising one or more of T-progenitor cells, precursor T cells, double positive T cells, single positive T cells, and regulatory T cells.

4-11. (canceled)

12. The method of claim 1, wherein the PSC population is a human iPSC population derived from lymphocytes, cord blood cells, peripheral blood mononuclear cells, CD34+ cells, or human primary tissues.

13. (canceled)

14. The method of claim 12, wherein the iPSCs are homozygous for one or more HLA Class I and/or Class II genes.

15. (canceled)

16. The method of claim 12, wherein the iPSCs are gene-edited to delete one or more HLA Class I genes, delete one or more Class II genes, and/or delete one or more genes governing HLA or MHC expression or presentation capacity.

17-19. (canceled)

20. The method of claim 14, wherein the iPSCs comprise a deletion of HLA-A, are homozygous for both HLA-B and HLA-C, comprise a deletion of HLA-DPB1 and HLA-DQB1, and are homozygous for HLA-DRB1.

21. (canceled)

22. The method of claim 1, wherein the endothelial-to-hematopoietic transition generates an HSC and/or HSPC population comprising long-term hematopoietic stem cells (LT-HSCs), short-term hematopoietic stem cells (ST-HSCs), and HSPCs.

23-29. (canceled)

30. The method of claim 1, wherein induction of endothelial-to-hematopoietic transition comprises Piezo1 activation and wherein the Piezo1 activation is by contacting the CD34+-enriched cells or fraction thereof with one or more Piezo1 agonists.

31-32. (canceled)

33. The method of claim 1, wherein the HSC and/or HSPC population or fraction thereof is cultured with a partial or full Notch ligand to produce a population comprising CD7+ progenitor T cells or a derivative cell population.

34. The method of claim 33, wherein the CD7+ progenitor T cells do not express CD34 or express a diminished level of CD34 compared to the HSC population; and wherein the CD7+ progenitor T cells express CD5.

35-44. (canceled)

45. The method of claim 33, comprising generating regulatory T cells (Tregs) from the progenitor T cells or αβ CD3+ and/or CD4+ T cells generated therefrom.

46. The method of claim 45, wherein the regulatory T cells express CD8 and/or CD4.

47. The method of claim 45, wherein differentiation to Tregs comprises modifying Treg precursors to express FOXP3.

48. (canceled)

49. The method of claim 47, wherein the cells modified to express FOXP3 are the iPSCs, the CD34+ cells before EHT, the CD34+ cells after EHT, progenitor T cells, CD4+/CD8+ T cells, CD8+ αβ T cells, or CD4+ αβ T cells.

50. (canceled)

51. A cell composition comprising a T cell population or progenitor T cell population that is HLA-A^{neg}, HLA-DPB1^{neg}, and HLA-DQB1^{neg}.

52. The cell composition of claim 51, wherein the T cell population is homozygous for both HLA-B and HLA-C.

53. (canceled)

54. The cell composition of claim 51, wherein the T cell population is a T-progenitor cell population, a cytotoxic T cell (CTL) population, a helper T cell population, a Treg population, or a T cell population that expresses a chimeric antigen receptor (CAR).

55-60. (canceled)

61. A method for cell therapy, comprising administering the T cell population or progenitor T cell population of claim 5, to a human subject in need thereof.

62-64. (canceled)

65. The method of claim 61, wherein the human subject is a tissue or organ transplant recipient, and the T cells are regulatory T cells.

66. The method of claim 65, wherein the human subject is a recipient of an allogeneic organ or tissue transplant.

67-72. (canceled)


SEQUENCE LISTING (33 sequences)

SEQ ID NO: 1 - RNA, 20 nt, synthetic construct
  source 1..20: mol_type = other RNA
cgtagcccacggcgatgaag

SEQ ID NO: 2 - RNA, 20 nt, synthetic construct
  source 1..20: mol_type = other RNA
atttcttcacatccgtgtcc

SEQ ID NO: 3 - RNA, 20 nt, synthetic construct
  source 1..20: mol_type = other RNA
gtctcctggtcccaatactc

SEQ ID NO: 4 - RNA, 20 nt, synthetic construct
  source 1..20: mol_type = other RNA
gtagcccacggcgatgaagc

SEQ ID NO: 5 - RNA, 20 nt, synthetic construct
  source 1..20: mol_type = other RNA
tagcccacggcgatgaagcg

SEQ ID NO: 6 - RNA, 20 nt, synthetic construct
  source 1..20: mol_type = other RNA
gagggttcggggcgccatga

SEQ ID NO: 7 - RNA, 20 nt, synthetic construct
  source 1..20: mol_type = other RNA
ccggaacacacggaatgtga

SEQ ID NO: 8 - RNA, 20 nt, synthetic construct
  source 1..20: mol_type = other RNA
acagcgacgccgcgagccag

SEQ ID NO: 9 - RNA, 20 nt, synthetic construct
  source 1..20: mol_type = other RNA
ccactcacagattgaccgag

SEQ ID NO: 10 - RNA, 20 nt, synthetic construct
  source 1..20: mol_type = other RNA
cgacgccgcgagccagagga

SEQ ID NO: 11 - RNA, 20 nt, synthetic construct
  source 1..20: mol_type = other RNA
cagattgaccgagtggacct

SEQ ID NO: 12 - RNA, 20 nt, synthetic construct
  source 1..20: mol_type = other RNA
cagactgaccgagtggacct

SEQ ID NO: 13 - RNA, 20 nt, synthetic construct
  source 1..20: mol_type = other RNA
acagactgaccgagtggacc

SEQ ID NO: 14 - RNA, 20 nt, synthetic construct
  source 1..20: mol_type = other RNA
gccgggccgggacacggatg

SEQ ID NO: 15 - RNA, 20 nt, synthetic construct
  source 1..20: mol_type = other RNA
ccaggagacacggaatgtga

SEQ ID NO: 16 - RNA, 20 nt, synthetic construct
  source 1..20: mol_type = other RNA
ccagtcacagactgaccgag

SEQ ID NO: 17 - RNA, 20 nt, synthetic construct
  source 1..20: mol_type = other RNA
tcgacagcgacgccgcgagc

SEQ ID NO: 18 - RNA, 20 nt, synthetic construct
  source 1..20: mol_type = other RNA
gagatacatctacaaccggg

SEQ ID NO: 19 - RNA, 20 nt, synthetic construct
  source 1..20: mol_type = other RNA
tggagagatacatctacaac

SEQ ID NO: 20 - RNA, 20 nt, synthetic construct
  source 1..20: mol_type = other RNA
ggttgtagatgtatctctcc

SEQ ID NO: 21 - RNA, 20 nt, synthetic construct
  source 1..20: mol_type = other RNA
ggagagatacatctacaacc

SEQ ID NO: 22 - RNA, 20 nt, synthetic construct
  source 1..20: mol_type = other RNA
aggaatgctacgcgtttaat

SEQ ID NO: 23 - RNA, 20 nt, synthetic construct
  source 1..20: mol_type = other RNA
cagatacatctataaccgag

SEQ ID NO: 24 - RNA, 20 nt, synthetic construct
  source 1..20: mol_type = other RNA
cgagtactggaacagccaga

SEQ ID NO: 25 - RNA, 20 nt, synthetic construct
  source 1..20: mol_type = other RNA
cccgttggtgaagtagcaca

SEQ ID NO: 26 - RNA, 20 nt, synthetic construct
  source 1..20: mol_type = other RNA
gtgctacttcaccaacggga

SEQ ID NO: 27 - RNA, 20 nt, synthetic construct
  source 1..20: mol_type = other RNA
aggtcgtgcggagctccaac

SEQ ID NO: 28 - RNA, 20 nt, synthetic construct
  source 1..20: mol_type = other RNA
aggtgacagttgctacccga

SEQ ID NO: 29 - RNA, 20 nt, synthetic construct
  source 1..20: mol_type = other RNA
tcccgttggtgaagtagcac

SEQ ID NO: 30 - RNA, 20 nt, synthetic construct
  source 1..20: mol_type = other RNA
aactacgaggtggcgtaccg

SEQ ID NO: 31 - RNA, 20 nt, synthetic construct
  source 1..20: mol_type = other RNA
ctgttccagtactcggcaac

SEQ ID NO: 32 - RNA, 20 nt, synthetic construct
  source 1..20: mol_type = other RNA
actacgaggtggcgtaccgc

SEQ ID NO: 33 - RNA, 20 nt, synthetic construct
  source 1..20: mol_type = other RNA
cgaagcgcacgtactcctct